SEQUENCE LISTING (70 sequences)

SEQ ID NO: 1 - DNA, 20 bp, synthetic construct
  misc_feature 1..20: note = Synthetic
  source 1..20: mol_type = other DNA
tgtggatcagcaagcaggag

SEQ ID NO: 2 - DNA, 20 bp, synthetic construct
  misc_feature 1..20: note = Synthetic
  source 1..20: mol_type = other DNA
tgcgcaagttaggttttgtc

SEQ ID NO: 3 - DNA, 23 bp, synthetic construct
  misc_feature 1..23: note = Synthetic
  source 1..23: mol_type = other DNA
ggtagaaaaagcaaccacgaagc

SEQ ID NO: 4 - DNA, 25 bp, synthetic construct
  misc_feature 1..25: note = Synthetic
  source 1..25: mol_type = other DNA
acataaacctctgtctgtgagtgcc

SEQ ID NO: 5 - DNA, 20 bp, synthetic construct
  misc_feature 1..20: note = Synthetic
  source 1..20: mol_type = other DNA
gaactttctgctgtcttggg

SEQ ID NO: 6 - DNA, 20 bp, synthetic construct
  misc_feature 1..20: note = Synthetic
  source 1..20: mol_type = other DNA
cttcgtgatgattctgccct

SEQ ID NO: 7 - DNA, 20 bp, synthetic construct
  misc_feature 1..20: note = Synthetic
  source 1..20: mol_type = other DNA
ctctctctcaaggccccaaa

SEQ ID NO: 8 - DNA, 20 bp, synthetic construct
  misc_feature 1..20: note = Synthetic
  source 1..20: mol_type = other DNA
agacttgggcctctgttacc

SEQ ID NO: 9 - DNA, 19 bp, synthetic construct
  misc_feature 1..19: note = Synthetic
  source 1..19: mol_type = other DNA
ggacacggcctatagcctg

SEQ ID NO: 10 - DNA, 20 bp, synthetic construct
  misc_feature 1..20: note = Synthetic
  source 1..20: mol_type = other DNA
ctcttggcgcagttcttgtc

SEQ ID NO: 11 - DNA, 20 bp, synthetic construct
  misc_feature 1..20: note = Synthetic
  source 1..20: mol_type = other DNA
tgtggatcagcaagcaggag

SEQ ID NO: 12 - DNA, 20 bp, synthetic construct
  misc_feature 1..20: note = Synthetic
  source 1..20: mol_type = other DNA
tgcgcaagttaggttttgtc

SEQ ID NO: 13 - DNA, 20 bp, synthetic construct
  misc_feature 1..20: note = Synthetic
  source 1..20: mol_type = other DNA
gcgccaacgtagaccaaaag

SEQ ID NO: 14 - DNA, 20 bp, synthetic construct
  misc_feature 1..20: note = Synthetic
  source 1..20: mol_type = other DNA
tctaccgatgttggggacct

SEQ ID NO: 15 - DNA, 20 bp, synthetic construct
  misc_feature 1..20: note = Synthetic
  source 1..20: mol_type = other DNA
atgcggatcaaacctcacca

SEQ ID NO: 16 - DNA, 21 bp, synthetic construct
  misc_feature 1..21: note = Synthetic
  source 1..21: mol_type = other DNA
ctttctttggtctgcattcac

SEQ ID NO: 17 - DNA, 20 bp, synthetic construct
  misc_feature 1..20: note = Synthetic
  source 1..20: mol_type = other DNA
cgctgtgtcccatcgtattg

SEQ ID NO: 18 - DNA, 20 bp, synthetic construct
  misc_feature 1..20: note = Synthetic
  source 1..20: mol_type = other DNA
agacttgggcctctgttacc

SEQ ID NO: 19 - DNA, 20 bp, synthetic construct
  misc_feature 1..20: note = Synthetic
  source 1..20: mol_type = other DNA
ttggtacctgtagccattcc

SEQ ID NO: 20 - DNA, 20 bp, synthetic construct
  misc_feature 1..20: note = Synthetic
  source 1..20: mol_type = other DNA
gaggctaagaggctgctgta

SEQ ID NO: 21 - RNA, 21 nt, synthetic construct
  misc_feature 1..21: note = Synthetic
  source 1..21: mol_type = other RNA
ccaaagagtctgatgtttaca

SEQ ID NO: 22 - RNA, 21 nt, synthetic construct
  misc_feature 1..21: note = Synthetic
  source 1..21: mol_type = other RNA
taaacatcagactctttgggg

SEQ ID NO: 23 - RNA, 21 nt, synthetic construct
  misc_feature 1..21: note = Synthetic
  source 1..21: mol_type = other RNA
cataaacaatatatctgttgg

SEQ ID NO: 24 - RNA, 21 nt, synthetic construct
  misc_feature 1..21: note = Synthetic
  source 1..21: mol_type = other RNA
aacagatatattgtttatgag

SEQ ID NO: 25 - RNA, 21 nt, synthetic construct
  misc_feature 1..21: note = Synthetic
  source 1..21: mol_type = other RNA
ctctataatatgcttatatta

SEQ ID NO: 26 - RNA, 21 nt, synthetic construct
  misc_feature 1..21: note = Synthetic
  source 1..21: mol_type = other RNA
atataagcatattatagagtt

SEQ ID NO: 27 - RNA, 21 nt, synthetic construct
  misc_feature 1..21: note = Synthetic
  source 1..21: mol_type = other RNA
gtgtatatataataatgtatt

SEQ ID NO: 28 - RNA, 21 nt, synthetic construct
  misc_feature 1..21: note = Synthetic
  source 1..21: mol_type = other RNA
tacattattatatatacacaa

SEQ ID NO: 29 - DNA, 23 bp, synthetic construct
  misc_feature 1..23: note = Synthetic
  source 1..23: mol_type = other DNA
ccccaaagagtctgatgtttaca

SEQ ID NO: 30 - DNA, 23 bp, synthetic construct
  misc_feature 1..23: note = Synthetic
  source 1..23: mol_type = other DNA
ctcataaacaatatatctgttgg

SEQ ID NO: 31 - DNA, 23 bp, synthetic construct
  misc_feature 1..23: note = Synthetic
  source 1..23: mol_type = other DNA
aactctataatatgcttatatta

SEQ ID NO: 32 - DNA, 23 bp, synthetic construct
  misc_feature 1..23: note = Synthetic
  source 1..23: mol_type = other DNA
ttgtgtatatataataatgtatt

SEQ ID NO: 33 - RNA, 21 nt, synthetic construct
  misc_feature 1..21: note = Synthetic
  source 1..21: mol_type = other RNA
taaagaaaacttcatttacag

SEQ ID NO: 34 - RNA, 21 nt, synthetic construct
  misc_feature 1..21: note = Synthetic
  source 1..21: mol_type = other RNA
gtaaatgaagttttctttatg

SEQ ID NO: 35 - RNA, 21 nt, synthetic construct
  misc_feature 1..21: note = Synthetic
  source 1..21: mol_type = other RNA
tatagaaaaattctttaagag

SEQ ID NO: 36 - RNA, 21 nt, synthetic construct
  misc_feature 1..21: note = Synthetic
  source 1..21: mol_type = other RNA
cttaaagaatttttctatagt

SEQ ID NO: 37 - RNA, 21 nt, synthetic construct
  misc_feature 1..21: note = Synthetic
  source 1..21: mol_type = other RNA
ttttaaaaataaatattggag

SEQ ID NO: 38 - RNA, 21 nt, synthetic construct
  misc_feature 1..21: note = Synthetic
  source 1..21: mol_type = other RNA
ccaatatttatttttaaaaaa

SEQ ID NO: 39 - RNA, 21 nt, synthetic construct
  misc_feature 1..21: note = Synthetic
  source 1..21: mol_type = other RNA
aattatatcacattcttacac

SEQ ID NO: 40 - RNA, 21 nt, synthetic construct
  misc_feature 1..21: note = Synthetic
  source 1..21: mol_type = other RNA
gtaagaatgtgatataattct

SEQ ID NO: 41 - DNA, 23 bp, synthetic construct
  misc_feature 1..23: note = Synthetic
  source 1..23: mol_type = other DNA
ctgtaaatgaagttttctttatg

SEQ ID NO: 42 - DNA, 23 bp, synthetic construct
  misc_feature 1..23: note = Synthetic
  source 1..23: mol_type = other DNA
ctcttaaagaatttttctatagt

SEQ ID NO: 43 - DNA, 23 bp, synthetic construct
  misc_feature 1..23: note = Synthetic
  source 1..23: mol_type = other DNA
ctccaatatttatttttaaaaaa

SEQ ID NO: 44 - DNA, 23 bp, synthetic construct
  misc_feature 1..23: note = Synthetic
  source 1..23: mol_type = other DNA
gtgtaagaatgtgatataattct

SEQ ID NO: 45 - RNA, 21 nt, synthetic construct
  modified_base 20..21: mod_base = OTHER; note = thymine
  misc_feature 1..21: note = Synthetic
  source 1..21: mol_type = other RNA
ggagtaccctgatgagatctt

SEQ ID NO: 46 - RNA, 21 nt, synthetic construct
  modified_base 20..21: mod_base = OTHER; note = thymine
  misc_feature 1..21: note = Synthetic
  source 1..21: mol_type = other RNA
gatctcatcagggtactcctt

SEQ ID NO: 47 - RNA, 21 nt, synthetic construct
  misc_feature 1..21: note = Synthetic
  source 1..21: mol_type = other RNA
ctgttaagaaaaatctagaaa

SEQ ID NO: 48 - RNA, 21 nt, synthetic construct
  misc_feature 1..21: note = Synthetic
  source 1..21: mol_type = other RNA
tctagatttttcttaacagct

SEQ ID NO: 49 - DNA, 20 bp, synthetic construct
  misc_feature 1..20: note = Synthetic
  source 1..20: mol_type = other DNA
ttccagcacctggacaatga

SEQ ID NO: 50 - DNA, 20 bp, synthetic construct
  misc_feature 1..20: note = Synthetic
  source 1..20: mol_type = other DNA
acagacaaccaccagcaaca

SEQ ID NO: 51 - DNA, 20 bp, synthetic construct
  misc_feature 1..20: note = Synthetic
  source 1..20: mol_type = other DNA
gagagtgaccaccatcagct

SEQ ID NO: 52 - DNA, 20 bp, synthetic construct
  misc_feature 1..20: note = Synthetic
  source 1..20: mol_type = other DNA
tcgctgtgaagttgctgaac

SEQ ID NO: 53 - RNA, 21 nt, synthetic construct
  misc_feature 1..21: note = Synthetic
  source 1..21: mol_type = other RNA
aaatgtttctaataattccat

SEQ ID NO: 54 - RNA, 21 nt, synthetic construct
  misc_feature 1..21: note = Synthetic
  source 1..21: mol_type = other RNA
ggaattattagaaacattttc

SEQ ID NO: 55 - RNA, 21 nt, synthetic construct
  misc_feature 1..21: note = Synthetic
  source 1..21: mol_type = other RNA
ttattcttctcatcaaaaggg

SEQ ID NO: 56 - RNA, 21 nt, synthetic construct
  misc_feature 1..21: note = Synthetic
  source 1..21: mol_type = other RNA
cttttgatgagaagaataagc

SEQ ID NO: 57 - RNA, 21 nt, synthetic construct
  misc_feature 1..21: note = Synthetic
  source 1..21: mol_type = other RNA
tctgatttgcaaaaacagctg

SEQ ID NO: 58 - RNA, 21 nt, synthetic construct
  misc_feature 1..21: note = Synthetic
  source 1..21: mol_type = other RNA
gctgtttttgcaaatcagatt

SEQ ID NO: 59 - RNA, 21 nt, synthetic construct
  misc_feature 1..21: note = Synthetic
  source 1..21: mol_type = other RNA
taaagtagaccaactaagcga

SEQ ID NO: 60 - RNA, 21 nt, synthetic construct
  misc_feature 1..21: note = Synthetic
  source 1..21: mol_type = other RNA
gcttagttggtctactttaaa

SEQ ID NO: 61 - RNA, 21 nt, synthetic construct
  misc_feature 1..21: note = Synthetic
  source 1..21: mol_type = other RNA
ctgttaagaaaaatctagaaa

SEQ ID NO: 62 - RNA, 21 nt, synthetic construct
  misc_feature 1..21: note = Synthetic
  source 1..21: mol_type = other RNA
ttaagtagttgtattaatctc

SEQ ID NO: 63 - RNA, 21 nt, synthetic construct
  misc_feature 1..21: note = Synthetic
  source 1..21: mol_type = other RNA
gattaatacaactacttaaaa

SEQ ID NO: 64 - DNA, 23 bp, synthetic construct
  misc_feature 1..23: note = Synthetic
  source 1..23: mol_type = other DNA
atggaattattagaaacattttc

SEQ ID NO: 65 - DNA, 23 bp, synthetic construct
  misc_feature 1..23: note = Synthetic
  source 1..23: mol_type = other DNA
cccttttgatgagaagaataagc

SEQ ID NO: 66 - DNA, 23 bp, synthetic construct
  misc_feature 1..23: note = Synthetic
  source 1..23: mol_type = other DNA
cagctgtttttgcaaatcagatt

SEQ ID NO: 67 - DNA, 23 bp, synthetic construct
  misc_feature 1..23: note = Synthetic
  source 1..23: mol_type = other DNA
tcgcttagttggtctactttaaa

SEQ ID NO: 68 - DNA, 23 bp, synthetic construct
  misc_feature 1..23: note = Synthetic
  source 1..23: mol_type = other DNA
agctgttaagaaaaatctagaaa

SEQ ID NO: 69 - DNA, 23 bp, synthetic construct
  misc_feature 1..23: note = Synthetic
  source 1..23: mol_type = other DNA
gagattaatacaactacttaaaa

SEQ ID NO: 70 - RNA, 21 nt, synthetic construct
  misc_feature 1..21: note = Synthetic
  source 1..21: mol_type = other RNA
tctagatttttcttaacagct

(19) United States
(12) Patent Application Publication
(10) Pub. No.: US 2026/0250678 A1
(43) Pub. Date: Aug. 27, 2026

(54) Novel treatment strategies for diabetic retinopathy and other ocular diseases

(71) Applicant: The University of Western Ontario, London (CA)

(72) Inventors: Subrata CHAKRABARTI, London (CA); Biao FENG, London (CA); Eric WANG, London (CA); Shali CHEN, London (CA); Hong Lin WANG, JR., London (CA)

(21) Appl. No.: 19/454,863

(22) Filed: Jan. 21, 2026

Related U.S. Application Data
(63) Continuation of application No. PCT/CA2025/050233, filed on Feb. 24, 2025.
(60) Provisional application No. 63/556,525, filed on Feb. 22, 2024.
(60) Provisional application No. 63/710,463, filed on Oct. 22, 2024.

(51) Int. Cl. C12N 15/113; A61P 27/02

(52) U.S. Cl. CPC C12N 15/113; A61P 27/02; C12N 2310/14; C12N 2310/351; C12N 2310/3513; C12N 2320/31; C12N 2320/32

(57) ABSTRACT

A method of treating or preventing an ocular disease in a subject comprising administering locally to an eye of the subject a nucleic acid (NA) molecule having an acetylated amino sugar moiety (CHO) (CHO-NA), wherein the NA is an inhibitor of at least one biological activity in the eye associated with the ocular disease and is effective in the treatment or prevention of the ocular disease. Also a composition comprising a CHO-NA for the treatment of an ocular disease. In embodiments, the CHO is an N-galactosamine moiety.

### CROSS-REFERENCE TO RELATED APPLICATIONS

This application is a continuation of International Patent Application No. PCT/US2025/050233 filed on Feb. 24, 2025, which claims the benefit of priority U.S. Provisional Patent Application Nos. 63/556,525, filed Feb. 22, 2024, and 63/710,463, filed Oct. 22, 2024. The disclosure of the above-referenced applications is herein expressly incorporated by reference in their entirety, including any drawings.

### INCORPORATION BY REFERENCE OF SEQUENCE LISTING

This application contains an accompanying Sequence Listing, which is incorporated by reference in its entirety. The accompanying Sequence Listing is provided in electronic format as an XML file, named, “065213-502C01US_PCTCA2025050233-seql-000001-EN-20250414,” which was created on Apr. 3, 2025 and is 90,026 bytes in size.

### FIELD OF TECHNOLOGY

The present disclosure relates to treatment and/or prevention strategies for diabetic retinopathy and/or other ocular diseases. Particularly, the present disclosure relates to GalNAc conjugated siRNAs for the treatment or prevention of diabetic retinopathy and/or other ocular diseases.

### BACKGROUND INFORMATION

Diabetes is a leading cause of mortality and reduced quality of life worldwide, and the prevalence of diabetes is projected to continue its astronomic rise, affecting well over 700 million people by 2045 (1,2). With the number of patients living with diabetes on the rise, the number of people afflicted with chronic diabetic complications will correspondingly see a significant increase. Diabetic retinopathy (DR) is one of the major chronic complications of diabetes and is a leading cause of vision impairment in working-aged adults (3-7). DR can be broadly separated into two stages, a vaso-obliterative stage known as non-proliferative diabetic retinopathy (NPDR), and a vaso-proliferative stage known as proliferative diabetic retinopathy (PDR) (7-12). Vision impairment can occur at either stage through a process known as diabetic macular edema (DME), where the blood vessels of the eye become leaky following hyperglycemic insult, causing fluid to accumulate in pockets in the retina, leading to visual distortion (6-12). Furthermore, in PDR there is another mechanism for vision loss known as tractional retinal detachment (TRD), where newly synthesized vessels create traction, which pull on the retina until it detaches from the back of the eye (7-12).

Central to the pathogenesis of DR, as well as other chronic diabetic complications, are hyperglycemia-induced changes to the microvascular blood vessels (7-12). These changes are brought about by aberrant activation of pro-angiogenic signal cascades following hyperglycemic stress, leading to the release of various angiogenic factors, key among which is vascular endothelial growth factor A (VEGF)(7-12). VEGF is expressed by many cell types and binds to receptors on the surface of endothelial cells to induce angiogenesis and increase vascular permeability (13). Given its central importance in DR, many first-line therapies target the VEGF signaling pathway (12-14). These therapeutics are injected into the eye and work by sequestering VEGF to prevent its actions, but they are limited in various ways (12-14). Firstly, the frequent intraocular injections are invasive and a burden for the patient. Secondly, because VEGF is expressed in varying quantities by healthy organs as part of normal biological processes, anti-VEGF therapy, which indiscriminately inhibits VEGF, can cause significant adverse effects, either locally or systemically (12-15). Furthermore, some patients with DR either do not respond at all or fail to fully respond to anti-VEGF treatments, resulting in primary or secondary failure in up to 50% of patients (12,15). Finally, despite VEGF being one of the main angiogenic factors at play in vision-threatening DR, it is not the only one, therefore even if VEGF signaling in the retina can be fully blocked, there remains other avenues for disease progression (7, 10-12) All of these limitations, taken together, undoubtedly necessitates the urgent creation and adoption of a more robust, more effective and less invasive solution to vision loss in DR.

DR is complex and multifaceted in its pathology; hence it requires more than the “blunt instruments” that anti-VEGF therapies represent. DR begins with cellular changes that accumulate over time to produce tissue-level defects (7-12). Long non-coding RNAs (lncRNAs) are known as epigenetic regulators of disease and influence intracellular changes through multiple mechanisms at various levels (16-18). Epigenetics is the study of non-genomic mechanisms behind persistent intracellular changes, and epigenetic regulators are mediators of such changes. lncRNA-based therapy for DR can be longer-lasting than current approaches, because lncRNAs help reprogram cells at the root of the problem (16-22). Furthermore, a single lncRNA can influence various intracellular processes, producing a more robust, multifaceted effect than current anti-VEGF approaches (16-22).

Age-related macular degeneration (AMD) is a progressive age-related disease that affects 1 in 10 people over 50 years of age, and 1 in 3 people over the age of 80 (47) and the risk of AMD is increased by diabetes (50). AMD is broadly categorized into 2 types, atrophic (dry) AMD, and neovascular (wet) AMD. Dry AMD can be subclassified into stages and can progress into wet AMD from any of its stages. Neovascularization in wet AMD is driven by the excessive production and release of angiogenic factors such as VEGF-A and is typically treated using intraocular injections of anti-VEGF antibodies (48). However, as with diabetic retinopathy, some patients with AMD are also non-responsive to anti-VEGF antibody treatments, and many patients develop resistance to such treatments (48). As with diabetic retinopathy, patient non-response and resistance are likely also due to the fact that multiple angiogenic factors are involved in neovascularization in wet AMD.

The pathophysiological processes of vision impairment via neovascularization are substantially similar amongst ocular diseases such as diabetic retinopathy (DR_ and age-related macular degeneration (AMD), and current treatment approaches to neovascularization in both DR and AMD rely heavily on anti-VEGF antibodies. However, it is evident in both DR and AMD, that targeting only VEGF is often inadequate to fully address neovascularization (48, 49). Regulatory lncRNAs are important in the pathogenesis and progression of AMD (49).

### SUMMARY OF DISCLOSURE

In one embodiment, the present disclosure provides a method of treating or preventing an ocular disease in a subject comprising administering locally to an eye of the subject a nucleic acid molecule having a carbohydrate moiety (CHO) (CHO-NA), wherein the CHO-NA is effective in the treatment or prevention of the ocular disease. In one aspect, the carbohydrate moiety is an acetylated amino sugar moiety.

In one embodiment of the method of treating or preventing an ocular disease of the present disclosure, the NA is a small interfering RNA (siRNA), a microRNA, a PIWI-interacting RNA (piRNA), a small nuclear RNA, an antisense nucleic acid, short hair-pin RNA (shRNA) or a ribozyme.

In another embodiment of the method of treating or preventing an ocular disease of the present disclosure, the NA is a siRNA (CHO-siRNA).

In another embodiment of the method of treating or preventing an ocular disease of the present disclosure, the siRNA in the CHO-siRNA is one or more of a HOTAIR siRNA (CHO-siHOTAIR), a VEGFA siRNA (CHO-siVEGFA), a MALAT1 siRNA (CHO-siMALAT1), an ANRIL siRNA (CHO-siANRIL), a WISPER siRNA (CHO-siWISPER), a H19 siRNA (CHO-siH19), a MIAT siRNA (CHO-siMIAT) and ZFAS1 siRNA (CHO-siZFAS1).

In another embodiment of the method of treating or preventing an ocular disease of the present disclosure, the NA is an inhibitor of a long-noncoding RNA (lncRNA) associated with the ocular disease.

In another embodiment of the method of treating or preventing an ocular disease of the present disclosure, the lncRNA is one or more of HOTAIR lncRNA, MALAT1 lncRNA, ANRIL lncRNA, WISPER lncRNA, H19 lncRNA, MIAT lncRNAZFAS1 lncRNA.

In another embodiment of the method of treating or preventing an ocular disease of the present disclosure, the CHO is an N-acetylgalactosamine (GalNAc).

In another embodiment of the method of treating or preventing an ocular disease of the present disclosure, the CHO-NA is a HOTAIR siRNA having an N-acetylgalactosamine (GalNAc-siHOTAIR), and wherein the HOTAIR siRNA (siHOTAIR) comprises a sequence selected from SEQ ID NO: 22, SEQ ID NO: 24, SEQ ID NO: 26, and SEQ ID NO:28, or wherein the siHOTAIR comprises a sequence selected from SEQ ID NO: 21, SEQ ID NO: 23, SEQ ID NO: 25, and SEQ ID NO:27.

In another embodiment of the method of treating or preventing an ocular disease of the present disclosure, the local administration is one of topical administration, subretinal injection, subconjunctival injection, intravitreal injection, or a combination thereof.

In another embodiment of the method of treating or preventing an ocular disease of the present disclosure, the topical administration includes eye drops and hydrogels.

In another embodiment of the method of treating or preventing an ocular disease of the present disclosure, the local administration of the CHO-NA is free of a transfection agent.

In another embodiment of the method of treating or preventing an ocular disease of the present disclosure, the CHO-NA is administered in combination with (i) another different CHO-NA, (ii) in combination with another agent known to treat or prevent the ocular disease, or (iii) in combination with both (i) and (ii).

In another embodiment of the method of treating or preventing an ocular disease of the present disclosure, the ocular disease is an eye-related diabetes-induced condition.

In another embodiment of the method of treating or preventing an ocular disease of the present disclosure, ocular disease is a retinal disease.

In another embodiment of the method of treating or preventing an ocular disease of 20 the present disclosure, the ocular disease includes dry age-related macular degeneration (AMD), wet AMD, non-proliferative diabetic retinopathy, proliferative diabetic retinopathy, neovascular glaucoma and/or ischemic retinopathy.

In another embodiment of the method of treating or preventing an ocular disease of the present disclosure, the CHO is a ligand of a retinal asialoglycoprotein receptor (ASGR).

In another embodiment, the present disclosure relates to a nucleic acid molecule (NA) having a carbohydrate moiety (CHO) (CHO-NA) for local use in treating or preventing an ocular disease. In one aspect, the carbohydrate moiety is an acetylated amino sugar moiety.

In one embodiment of the NA for local use of the present disclosure, the NA is a small interfering RNA (siRNA), a microRNA, a PIWI-interacting RNA (piRNA), a small nuclear RNA (snRNA), a short hair-pin RNA (shRNA), an antisense nucleic acid or a ribozyme.

In another embodiment of the NA for local use of the present disclosure, the NA is a siRNA (CHO-siRNA).

In another embodiment of the NA for local use of the present disclosure, the CHO-siRNA is one or more of a HOTAIR sIRNA (CHO-siHOTAIR), a VEGFA siRNA (CHO-siVEGFA), a MALAT1 siRNA (CHO-siMALAT1), a ANRIL siRNA (CHO-siANRIL), a WISPER siRNA (CHO-siWISPER), a H19 siRNA (CHO-siH19), a MIAT siRNA (CHO-siMIAT) and ZFAS1 siRNA (CHO-siZFAS1).

In another embodiment of the NA for local use of the present disclosure, the NA is an inhibitor of a long-noncoding RNA (lncRNA) associated with the ocular disease.

In another embodiment of the NA for local use of the present disclosure, the lncRNA is one or more of HOTAIR lncRNA, MALAT1 lncRNA, ANRIL lncRNA, WISPER lncRNA, H19 lncRNA, MIAT lncRNA and ZFAS1 lncRNA.

In another embodiment of the NA for local use of the present disclosure, the CHO is an N-acetylgalactosamine (GalNAc).

In another embodiment of the NA for local use of the present disclosure, the CHO is N-acetylgalactosamine (GalNAc) and the siRNA is a HOTAIR siRNA (siHOTAIR), and wherein the siHOTAIR comprises a sequence selected from SEQ ID NO: 22, SEQ ID NO: 24, SEQ ID NO: 26, and SEQ ID NO:28, or wherein the siHOTAIR comprises a sequence selected from SEQ ID NO: 21, SEQ ID NO: 23, SEQ ID NO: 25, and SEQ ID NO:27.

In another embodiment of the NA for local use of the present disclosure, the CHO-NA is provided in the form of a subretinal injection, a subconjunctival injection, an intravitreal injection, eye drops, hydrogels or a combination thereof.

In another embodiment of the NA for local use of the present disclosure, the local use of the CHO-NA is free of a transfection agent.

In another embodiment of the NA for local use of the present disclosure, the CHO-NA is used in combination with (i) another different CHO siRNA, (ii) in combination with another agent known to treat or prevent the ocular disease, or (iii) in combination with both (i) and (ii).

In another embodiment of the NA for local use of the present disclosure, the ocular disease is an eye-related diabetes-induced condition.

In another embodiment of the NA for local use of the present disclosure, the ocular disease is a retinal disease.

In another embodiment of the NA for local use of the present disclosure, the ocular disease is dry age-related macular degeneration (AMD), wet AMD, non-proliferative diabetic retinopathy, proliferative diabetic retinopathy, neovascular glaucoma and/or ischemic retinopathy.

In another embodiment of the NA for local use of the present disclosure, the CHO is a ligand of a retinal asialoglycoprotein receptor (ASGR).

In another embodiment, the present disclosure relates to a use of a nucleic acid (NA) molecule having a carbohydrate moiety (CHO) (CHO-NA) in the manufacture of a medicament for local treatment or prevention of an ocular disease. In one aspect, the carbohydrate moiety is an acetylated amino sugar moiety.

In one embodiment of the use of the CHO-NA in the manufacture of a medicament of the present disclosure, the NA is a small interfering RNA (siRNA), a microRNA, a PIWI-interacting RNA (piRNA), a small nuclear RNA (snRNA), a short hair-pin RNA (shRNA), an antisense nucleic acid or a ribozyme.

In one embodiment of the use of the CHO-NA in the manufacture of a medicament of the present disclosure, the NA is a siRNA (CHO-siRNA).

In another embodiment of the use of the CHO-NA in the manufacture of a medicament of the present disclosure, the siRNA in the CHO-siRNA is one or more of a HOTAIR siRNA (CHO-siHOTAIR), a VEGFA siRNA (CHO-siVEGFA), MALAT1 siRNA (CHO-siMALAT1), a ANRIL siRNA (CHO-siANRIL), a WISPER siRNA (CHO-siWISPER), a H19 siRNA (CHO-siH19), a MIAT siRNA (siMIAT) and ZFAS1 siRNA (CHO-siZFAS1).

In another embodiment of the use of the CHO-NA in the manufacture of a medicament of the present disclosure, the NA is an inhibitor of a long-noncoding RNA (lncRNA) associated with the ocular disease.

In another embodiment of the use of the CHO-NA in the manufacture of a medicament of the present disclosure, the lncRNA is one or more of HOTAIR lncRNA, MALAT1 lncRNA, ANRIL lncRNA, WISPER lncRNA, H19 lncRNA, MIAT lncRNA and ZFAS1 lncRNA.

In another embodiment of the use of the CHO-NA in the manufacture of a medicament of the present disclosure, the CHO is a GalNAc.

In another embodiment of the use of the CHO-NA in the manufacture of a medicament of the present disclosure, the CHO is a GalNAc and the NA is a HOTAIR siRNA (siHOTAIR), and wherein the siHOTAIR comprises an sequence selected from SEQ ID NO: 22, SEQ ID NO: 24, SEQ ID NO: 26, and SEQ ID NO:28, or wherein the siHOTAIR comprises a sequence selected from SEQ ID NO: 21, SEQ ID NO: 23, SEQ ID NO: 25, and SEQ ID NO:27.

In another embodiment of the use of the CHO-NA in the manufacture of a medicament of the present disclosure, the medicament is formulated as a subretinal injection, a subconjunctival injection, an intravitreal injection, eye drops, hydrogels or a combination thereof.

In another embodiment of the use of the CHO-NA in the manufacture of a medicament of the present disclosure, the medicament is devoid of a transfection agent.

In another embodiment of the use of the CHO-NA in the manufacture of a medicament of the present disclosure, the medicament is used in combination with (i) another different CHO-NA, (ii) in combination with another agent known to treat or prevent the ocular disease, or (iii) in combination with both (i) and (ii).

In another embodiment of the use of the CHO-NA in the manufacture of a medicament of the present disclosure, the ocular disease is an eye-related diabetes-induced condition.

In another embodiment of the use of the CHO-NA in the manufacture of a medicament of the present disclosure, the ocular disease is a retinal disease.

In another embodiment of the use of the CHO-NA in the manufacture of a medicament of the present disclosure, the ocular disease is dry age-related macular degeneration (AMD), wet AMD, non-proliferative diabetic retinopathy, proliferative diabetic retinopathy, neovascular glaucoma and/or ischemic retinopathy.

In another embodiment of the use of the CHO-NA in the manufacture of a medicament of the present disclosure, the CHO is a ligand of a retinal asialoglycoprotein receptor (ASGR).

In another embodiment, the present disclosure relates to an eyedrop medicament comprising a nucleic acid molecule having a carbohydrate moiety (CHO) (CHO-NA).

In one aspect, the carbohydrate moiety is an acetylated amino sugar moiety.

In one embodiment of the eyedrop of the present disclosure, the NA is a small interfering RNA (siRNA), a microRNA, a PIWI-interacting RNA (piRNA), a short hair-pin RNA (shRNA), a small nuclear RNA (snRNA), an antisense nucleic acid or a ribozyme.

In another embodiment of the eyedrop of the present disclosure, the NA is a siRNA (CHO-siRNA).

In another embodiment of the eyedrop of the present disclosure, the siRNA is one or more of a HOTAIR siRNA (CHO-siHOTAIR), a VEGFA siRNA (CHO-siVEGFA), a MALAT1 siRNA (CHO-siMALAT1), an ANRIL siRNA (CHO-siANRIL), a WISPER siRNA (CHO-siWISPER), a H19 siRNA (CHO-siH19), a MIAT siRNA (CHO-siMIAT) and ZFAS1 siRNA (CHO-siZFAS1).

In another embodiment of the eyedrop of the present disclosure, the NA is an inhibitor of a long-noncoding RNA (lncRNA) associated with the ocular disease.

In another embodiment of the eyedrop of the present disclosure, the lncRNA is one or more of HOTAIR lncRNA, MALAT1 lncRNA, ANRIL lncRNA, WISPER lncRNA, H19 lncRNA, MIAT lncRNA and ZFAS1 lncRNA.

In another embodiment of the eyedrop of the present disclosure, the CHO is a GalNAc.

In another embodiment of the eyedrop of the present disclosure, the CHO is GalNAc and the siRNA is a HOTAIR siRNA (siHOTAIR), and wherein the siHOTAIR comprises a sequence selected from SEQ ID NO: 22, SEQ ID NO: 24, SEQ ID NO: 26, and SEQ ID NO:28, or wherein the siHOTAIR comprises a sequence selected from SEQ ID NO: 21, SEQ ID NO: 23, SEQ ID NO: 25, and SEQ ID NO:27.

In another embodiment of the eyedrop of the present disclosure, the eyedrop medicament is devoid of a transfection agent.

In another embodiment of the eyedrop of the present disclosure, the eyedrop medicament is used in combination with (i) another different CHO-NA, (ii) in combination with another agent known to treat or prevent the ocular disease, or (iii) in combination with both (i) and (ii).

In another embodiment of the eyedrop of the present disclosure, the ocular disease is an eye-related diabetes-induced condition.

In another embodiment of the eyedrop of the present disclosure, the ocular disease is a retinal disease.

In another embodiment of the eyedrop of the present disclosure, the ocular disease is dry age-related macular degeneration (AMD), wet AMD, non-proliferative diabetic retinopathy, proliferative diabetic retinopathy, neovascular glaucoma and/or ischemic retinopathy.

In another embodiment of the eyedrop of the present disclosure, the CHO is a ligand of a retinal asialoglycoprotein receptor (ASGR).

In another embodiment, the present disclosure provides an ocular injection comprising a nucleic acid molecule having a carbohydrate moiety (CHO) at one end (CHO-NA).

In one aspect, the carbohydrate moiety is an acetylated amino sugar moiety.

In one embodiment of the ocular injection of the present disclosure, the NA is a small interfering RNA (siRNA), a microRNA, a PIWI-interacting RNA (piRNA), a small nuclear RNA (snRNA), a short hair-pin RNA (shRNA), an antisense nucleic acid or a ribozyme.

In another embodiment of the ocular injection of the present disclosure, the NA is a siRNA (GalNAc-siRNA).

In another embodiment of the ocular injection of the present disclosure, the siRNA is one or more of a HOTAIR siRNA (CHO-siHOTAIR), a VEGFA siRNA (CHO-siVEGFA), a MALAT1 siRNA (CHO-siMALAT1), an ANRIL siRNA (CHO-siANRIL), a WISPER siRNA (CHO-siWISPER), H19 siRNA, a MIAT siRNA and a ZFAS1 siRNA (CHO-siZFAS1).

In another embodiment of the ocular injection of the present disclosure, the NA is an inhibitor of a long-noncoding RNA (lncRNA) associated with the ocular disease.

In another embodiment of the ocular injection of the present disclosure, the lncRNA is one or more of HOTAIR lncRNA, MALAT1 lncRNA, ANRIL lncRNA, WISPER lncRNA, H19 lncRNA, MIAT lncRNA and ZFAS1 lncRNA.

In another embodiment of the ocular injection of the present disclosure, the CHO is N-acetylgalactosamine (GalNAc).

In another embodiment of the ocular injection of the present disclosure, the CHO is GalNAc and the siRNA is siHOTAIR and wherein the siHOTAIR comprises a sequence selected from SEQ ID NO: 22, SEQ ID NO: 24, SEQ ID NO: 26, and SEQ ID NO:28, or wherein the siHOTAIR comprises a sequence selected from SEQ ID NO: 21, SEQ ID NO: 23, SEQ ID NO: 25, and SEQ ID NO:27.

In another embodiment of the ocular injection of the present disclosure, the ocular injection is devoid of a transfection agent.

In another embodiment of the ocular injection of the present disclosure, the ocular injection is used in combination with (i) another different CHO siRNA, (ii) in combination with another agent known to treat or prevent the ocular disease, or (iii) in combination with both (i) and (ii).

In another embodiment of the ocular injection of the present disclosure, the ocular disease is an eye-related diabetes-induced condition.

In another embodiment of the ocular injection of the present disclosure, the ocular disease is a retinal disease.

In another embodiment of the ocular injection of the present disclosure, the ocular disease is dry age-related macular degeneration (AMD), wet AMD, non-proliferative diabetic retinopathy, proliferative diabetic retinopathy, neovascular glaucoma and/or ischemic retinopathy.

In another embodiment of the ocular injection of the present disclosure, the CHO is a ligand of a retinal asialoglycoprotein receptor (ASGR).

In another embodiment, the present disclosure relates to a method of delivering a drug to the retina, the method comprising providing the drug linked to a ligand of a retinal asialoglycoprotein receptor (ASGR) and contacting the retina with the drug linked to the ligand.

In one embodiment of the method of delivering a drug to the retina of the present disclosure, the ligand is a carbohydrate moiety (CHO).

In another embodiment of the method of delivering a drug to the retina of the present disclosure, the CHO is an acetylated amino sugar moiety.

In another embodiment of the method of delivering a drug to the retina of the present disclosure, the CHO is N-acetylgalactosamine (GalNAc).

In another embodiment of the method of delivering a drug to the retina of the present disclosure, the drug is a nucleic acid molecule (NA).

In another embodiment of the method of delivering a drug to the retina of the present disclosure, the NA is a small interfering RNA (siRNA), a microRNA, a PIWI-interacting RNA (piRNA), a small nuclear RNA (snRNA), a short hair-pin RNA (shRNA), an antisense nucleic acid or a ribozyme.

In another embodiment of the method of delivering a drug to the retina of the present disclosure, the NA is an inhibitor of a long-noncoding RNA (lncRNA).

In another embodiment of the method of delivering a drug to the retina of the present disclosure, the lncRNA is one or more of HOTAIR lncRNA, MALAT1 lncRNA, ANRIL lncRNA, WISPER lncRNA, H19 lncRNA, MIAT lncRNA and ZFAS1 lncRNA.

In another embodiment of the method of delivering a drug to the retina of the present disclosure, the NA is one or more of HOTAIR siRNA, VEGFA siRNA, MALAT1 siRNA, ANRIL siRNA, WISPER siRNA, H19 siRNA, MIAT siRNA and ZFAS1 siRNA.

In another embodiment of the method of delivering a drug to the retina of the present disclosure, the contacting of the retina with the drug linked to the ligand is free of a transfection agent.

### BRIEF DESCRIPTION OF THE DRAWINGS

The following figures illustrate various aspects and preferred and alternative embodiments.

FIGS. 1A-1C—Chemical structure showing a multi-N-acetylgalactosamine (GalNAc) moiety covalently bound to an oligonucleotide molecule such as siRNA, optionally via a spacer molecule (linker). One or more than one GalNAc moieties (including modifications to GalNAc or GalNAc derivatives) can be covalently attached to one nucleic acid (NA) molecule, such as siRNA, thereby generating a cluster comprising 1, 2, or 3 GalNAc moieties (23, 24, 25). A GalNAc following the structure of FIG. 1A will be referred to in this document as “GalNAc [name of siRNA]” or as “GalNAc-modified [name of siRNA]-A”; a GalNAc following the structure of FIG. 1B will be referred to in this document as “GalNAc [name of siRNA]-B” or as “GalNAc-modified [name of siRNA]-B”; and a GalNAc following the structure of FIG. 1C will be referred to in this document as “GalNAc [name of siRNA]-C” or as “GalNAc-modified [name of siRNA]-C” Unless otherwise specified, the GalNAc-modified siRNA of FIG. 1A is used in the experiments described in the Examples.

FIG. 2—Graph showing GalNAc-modified siHOTAIR molecules preventing high glucose-mediated HOTAIR induction in human retinal endothelial cells without using transfection reagents (TR) in human retinal endothelial cells. Cells cultured under high glucose conditions showed significant glucose-induced upregulation of HOTAIR without any intervention. Treatment with each of the three GalNAC-modified siHOTAIRs (GalNAc siHOTAIR (FIG. 1A), GalNAc siHOTAIR-B (FIG. 1B), or GalNAc siHOTAIR-C (FIG. 10)) reduced HOTAIR expression in cells cultured in high glucose conditions. [NG=normal glucose (5 mM), HG=high glucose (25 mM); siRNA treatments given at 100 nM; lncRNA expression shown as ratios to the normal glucose condition; data normalized to [3-Actin mRNA and presented as mean+standard deviation; *=significantly different from all other groups.]

FIGS. 3A to 3B—Graphs showing GalNAc-modified siHOTAIR molecules preventing high glucose-mediated induction of angiogenic factors in human retinal endothelial cells without using transfection reagents (TR) in human retinal endothelial cells. Cells cultured under high glucose conditions showed significant glucose-induced upregulation of VEGFA (4A) and ANGPTL4 (4B) mRNA without any intervention. Treatment with each of the three GalNAC-modified siHOTAIRs (GalNAc siHOTAIR, GalNAc siHOTAIR-B, or GalNAc siHOTAIR-C) reduced VEGFA and ANGPTL4 mRNA expressions in cells cultured in high glucose conditions. [NG=normal glucose (5 mM), HG=high glucose (25 mM); siRNA treatments given at 100 nM; mRNA expressions shown as ratios to the normal glucose condition; data normalized to [3-Actin mRNA, and presented as mean+standard deviation; *=significantly different from all other groups.]

FIG. 4—Graph showing GalNAc modified siHOTAIR prevents high glucose-induced HOTAIR upregulation with or without transfection reagent in human retinal endothelial cells, while conventional siRNA only works with transfection reagent. Cells treated with scrambled oligonucleotides (SCR) or conventional siRNA without transfection reagent showed significant glucose-induced upregulation of HOTAIR. GalNAc siHOTAIR prevented high glucose-mediated HOTAIR induction without transfection reagent. When cells were treated with transfection reagent, only cells transfected with SCR had a high glucose-mediated induction of HOTAIR, while both conventional and GalNAc-modified siHOTAIR were able to prevent the upregulation. [NG=normal glucose (5 mM), HG=high glucose (25 mM); siRNA treatments given at 100 nM; lncRNA expression shown as ratios to the normal glucose condition; data normalized to p-Actin mRNA and presented as mean±standard deviation; *=significantly different, ns=not significant.]

FIG. 5—Graph showing GalNAc modified siHOTAIR prevents high glucose-induced VEGFA upregulation with or without transfection reagent in human retinal endothelial cells, while conventional siRNA only works with transfection reagent. Cells treated with scrambled oligonucleotides (SCR) or conventional siRNA without transfection reagent showed significant glucose-induced upregulation of VEGFA mRNA. GalNAc-modified siHOTAIR prevented high glucose-mediated VEGFA induction without transfection reagent. When cells were treated with transfection reagent, only cells transfected with SCR had a high glucose-mediated induction of VEGFA, while both conventional and GalNAc-modified siHOTAIR were able to prevent the upregulation. [NG=normal glucose (5 mM), HG=high glucose (25 mM); siRNA treatments given at 100 nM; lncRNA expression shown as ratios to the normal glucose condition; data normalized to [3-Actin mRNA and presented as mean±standard deviation; *=significantly different, ns=not significant.]

FIG. 6—Graph showing that GalNAc-modified siRNAs against regulatory lncRNAs is as effective at knocking down VEGFA mRNA as GalNAc-modified siRNAs designed specifically against VEGF mRNA. High glucose caused significant upregulation of VEGFA mRNA expression. Such upregulation was significantly reduced by GalNAc siVEGF, GalNAc siHOTAIR, and GalNAc siMALATA1. [siRNA treatments given at 100 nM; gene expression shown as ratios to the normal glucose condition; data normalized to [3-Actin mRNA and presented as mean+standard deviation; NG=normal glucose (5 mM), HG=high glucose (25 mM); *=significantly different from all other groups.]

FIG. 7—Graph showing that GalNAc-modified siHOTAIR effectively prevents high glucose-induced upregulation of HOTAIR at various concentrations in human retinal endothelial cells. High glucose treatment (HG; 25 mM) caused significant upregulation of HOTAIR compared to normal glucose (NG; 5 mM). GalNAc siHOTAIR was able to prevent the high glucose-induced HOTAIR upregulation at as low as 5 nM and showed similar efficacy up to 100 nM. [HOTAIR expressions shown as ratios to the NG condition; data are normalized to [3-Actin mRNA and presented as mean+standard deviation; *=significantly different from all other groups.]

FIG. 8—Graph showing that GalNAc-modified siHOTAIR effectively prevents high glucose-induced upregulation of VEGFA mRNA at various concentrations in human retinal endothelial cells. High glucose treatment (HG; 25 mM) caused significant upregulation of VEFGA mRNA compared to normal glucose (NG; 5 mM). GalNAc siHOTAIR was able to prevent the high glucose-induced VEGFA mRNA upregulation at as low as 5 nM and showed similar efficacy up to 100 nM. [VEGFA expressions shown as ratios to the NG condition; data are normalized to [3-Actin mRNA, and presented as mean+standard deviation; *=significantly different from all other groups.]

FIGS. 9A to 9B—Graphs illustrating high glucose (25 mM, HG) induced upregulation of HOTAIR long non-coding RNA (lncRNA) in human retinal endothelial cells was prevented by GalNAc-siHOTAIR without transfection reagent. The suppression of glucose-induced HOTAIR was visible at both day 4 (9A) and day 7 post-transfection (9B). The effects of GalNAc-siHOTAIR persisted for at least 7 days [NG=5 mM glucose; data are normalized to [3-Actin mRNA and presented as mean+standard deviation; *=significantly different from all other groups.]

FIG. 10—Graph showing high glucose (25 mM, HG) induced upregulation of VEGFA mRNA in human retinal endothelial cells were prevented by both conventional HOTAIR siRNA with transfection reagent and by GalNAc modified HOTAIR siRNA without transfection reagent. However, GalNAc siHOTAIR showed higher efficacy than Lucentis (an anti-VEGF prescription medication used for DR treatment). After 5 days, Modified Hotair-siRNA was still effective, but Lucentis was not (not shown). [NG=5 mM glucose; data are normalized to [3-Actin mRNA, and presented as mean+standard deviation; *=significantly different from NG+scrambled siRNA, **=significantly different from HG+scrambled siRNA, t=significantly different from HG+Lucentis.]

FIG. 11—Graph showing high glucose (25 mM, HG) induced upregulation of ANGPTL4 mRNA, another angiogenic mediator in retinopathy, in human retinal endothelial cells were prevented by both conventional HOTAIR siRNA with transfection reagent and by GalNAc modified HOTAIR siRNA without transfection reagent. However, GalNAc siHOTAIR showed much higher efficacy than Lucentis (Luc; an anti-VEGF prescription medication used for DR treatment). [NG=5 mM glucose, SCR=scrambled, Luc=Lucentis®. *=significantly different from NG+scrambled siRNA, **=significantly different from HG+scrambled siRNA, t=significantly different from HG+Lucentis.]

FIG. 12—Graph showing high glucose (25 mM, HG) induced upregulation of VEGFC mRNA, another member of the VEGF family, in human retinal endothelial cells. This upregulation was prevented by both conventional HOTAIR siRNA with transfection reagent and by GalNAc modified HOTAIR siRNA without transfection reagent. GalNAc siHOTAIR showed much higher efficacy than Lucentis (Luc; an anti-VEGF prescription medication used for DR treatment). [NG=5 mM glucose, SCR=scrambled, Luc=Lucentis®. *=significantly different from NG+scrambled siRNA, **=significantly different from HG+scrambled siRNA, t=significantly different from HG+Lucentis.]

FIGS. 13A to 13B—Graphs showing high glucose (25 mM, HG) induced upregulation of both VEGF-A protein (13A) and VEGF-C protein (13B) in human retinal endothelial cells were prevented by both conventional HOTAIR siRNA with transfection reagent and by GalNAc-modified HOTAIR siRNA without transfection reagent. [NG=5 mM glucose; data are presented as mean+standard deviation; *=significantly different from all other groups.]

FIGS. 14A to B—Graphs showing that GalNAc siHOTAIR prevented high glucose-mediated inhibition of miR-200b (14A) and miR-146a (14B) in human retinal endothelial cells. High glucose significantly reduces the level of the microRNA miR-200b and miR-146a (downregulation of miR-200b and miR-146a contributes to endothelial dysfunction in diabetic retinopathy). Treatment with GalNAc-modified siHOTAIR rescued the levels of both miR-200b and miR-146a in the human retinal endothelial cells. [siRNA treatment given at 100 nM; data presented as mean+standard deviation; NG=normal glucose (5 mM), HG=high glucose (25 mM), *= significantly different from all other groups.]

FIG. 15—Graph showing that GalNAc-modified siHOTAIR prevents high glucose-induced increase in permeability in human retinal endothelial cells. High glucose caused increased permeability in endothelial cells, as assessed via optical density (OD) reading at 450 nm using a trans-well permeability assay. Treatment with GalNAc siHOTAIR prevented high glucose-induced increase in vascular permeability. [siRNA treatments given at 100 nM; OD shown as ratios to the NG group; data presented as mean+standard deviation; *=significantly different from all other groups.]

FIGS. 16A to 16B—High glucose-induced angiogenesis is prevented by GalNAc-modified siHOTAIR. High glucose promoted angiogenesis, as seen via rapid formation of endothelial mesh networks in the in vitro tube formation assay (16A). Tube formation was quantified by analyzing the number of enclosed meshes formed in the duration of the assay (16B). GalNAc siHOTAIR suppressed high glucose-induced angiogenesis. 16A: Endothelial cells formed meshes on the basement membrane extract when exposed to high glucose. This effect was abrogated by treatment with GalNAc siHOTAIR. Microphotographs were taken at 4 hours after treatment, using a 10× objective lens. [siRNA treatments given at 100 nM; data presented as mean+standard deviation; NG=normal glucose (5 mM), HG=high glucose (25 mM); *=significantly different from all other groups.]

FIGS. 17A to 17F—Microphotographs showing that GalNAc modification helps target siRNA specifically to the retina. FAM-conjugated GalNAc-modified siRNAs were given topically to mice via eye drops (2 μL per drop, 2 drops per day for 5 consecutive days). The nuclear stain, DAPI shows abundant fluorescence in the cornea and lens (17A), ciliary body (17B), and retina (17C). FAM, tagged to GalNAc-modified siRNA, showed strong fluorescence only in the retina (17F), and not in the cornea and lens (17D), or ciliary body (17E), indicating specific localization of the GalNAc-modified siRNA to the retina. Original magnification 40× for 17A-17F.

FIGS. 18A to 18F—Microphotographs showing that treatment with GalNAc-modified siHOTAIR produced no histological evidence of toxicity in various organs in non-diabetic (not shown) or diabetic mice. Representative micrographs showing normal histologic appearance in the heart (18A), brain (18B), lung (18C), liver (18D), thymus (18E), or kidney (18F) following 5 consecutive days of eye drop treatments of diabetic mice. [Original magnification 25× for 18A-F.]

FIGS. 19A to 19B—Microphotographs demonstrating the inhibitory effect of GalNAc siHOTAIR on angiogenesis in mice. Matrigel plugs in mice with 500 ng VEGFA (VEGF) showed recruitment of ECs, an early sign of angiogenesis (19A). Plugs with the same quantity of VEGF but with the addition of GalNAc siHOTAIR (100 nM) without any transfection reagent showed reduced recruitment of ECs, and no signs of angiogenesis (19B). Although unmodified siRNA HOTAIR showed similar effects (not shown), it needed transfection with lipofectamine. [Original magnification 25× for both 19A and 19B.]

FIGS. 20A to 20B—Graphs showing that GalNAc siHOTAIR eyedrop reversed diabetes-induced upregulation of HOTAIR lncRNA (20A) and VEGFA mRNA (20B) in the retinas of diabetic mice. Whereas conventional siHOTAIR failed to produce any significant effects. Diabetic mice were treated with a drop of siHOTAIR (GalNAc or conventional) in one eye twice daily for five days. The other eye received mock treatments of distilled water. Retinal tissues were harvested and examined. No toxicity was noted. [Expression data shown as ratios to the non-diabetic condition; data are normalized to [3-Actin mRNA and presented as mean+standard deviation; *=significantly different from non-diabetic, **=significantly different from diabetic+eyedrop with conventional siHOTAIR.]

FIGS. 21A to 21B—Graphs showing that GalNAc siHOTAIR eyedrop reversed diabetes-induced upregulation of other important DR-related angiogenic factors ANGPTL4 mRNA (21A) and VEGFC mRNA (21B) in the retinas of diabetic mice. Whereas conventional siHOTAIR failed to produce any significant effects. Diabetic mice were treated with a drop of siHOTAIR (GalNAc or conventional) in one eye twice daily for five days. The other eye received mock treatments of distilled water. Retinal tissues were harvested and examined. No toxicity was noted. [Expression data shown as ratios to the non-diabetic condition; data are normalized to [3-Actin mRNA and presented as mean+standard deviation; *=significantly different from non-diabetic, **=significantly different from diabetic+eyedrop with conventional siHOTAIR.]

FIGS. 22A to 22B—Graphs showing that GalNAc siHOTAIR, delivered either via weekly injection or daily eye drops, reversed diabetes-induced upregulation of HOTAIR lncRNA (22A) and VEGFA mRNA (22B) in the retinas of diabetic mice. For injection treatment, diabetic mice were given 1 injection (1 μL) of GalNAc siHOTAIR and sacrificed one week following treatment. For eye drop treatment, diabetic mice were given 1 eye drop (2 μL) of GalNAc siHOTAIR per day for 1 week and then sacrificed. Retinal tissues were harvested and examined. No toxicity was noted. [lncRNA and mRNA expression data shown as ratios to the non-diabetic condition; data are normalized to [3-Actin mRNA and presented as mean+standard deviation; *=significantly different from all other groups.]

FIGS. 23A to 23B—Graphs showing that GalNAc siHOTAIR, delivered either via weekly injection or daily eye drops, reversed diabetes-induced upregulation of VEGFC (23A) and ANGPTL4 (23B) mRNAs in the retinas of diabetic mice. For injection treatment, diabetic mice were given 1 injection (1 μL) of GalNAc siHOTAIR and sacrificed one week following treatment. For eye drop treatment, diabetic mice were given 1 eye drop (2 μL) of GalNAc siHOTAIR per day for 1 week and then sacrificed. Retinal tissues were harvested and examined. No toxicity was noted. [lncRNA and mRNA expression data shown as ratios to the non-diabetic condition; data are normalized to [3-Actin mRNA and presented as mean+standard deviation; *=significantly different from all other groups.]

FIGS. 24A to 24B—Graphs showing that GalNAc siHOTAIR, delivered either via weekly injection or daily eye drops, reversed diabetes-induced upregulation of HOTAIR lncRNA (24A) and VEGFA mRNA (24B) in the retinas of diabetic rats. For injection treatment, diabetic rats were given 1 injection (10 μL) of GalNAc siHOTAIR and sacrificed one week following treatment. For eye drop treatment, diabetic rats were given 1 eye drop (5 μL) of GalNAc siHOTAIR per day for 1 week and then sacrificed. Retinal tissues were harvested and examined. No toxicity was noted. [lncRNA and mRNA expression data shown as ratios to the non-diabetic condition; data are normalized to [3-Actin mRNA and presented as mean+standard deviation; *=significantly different from all other groups.]

FIGS. 25A to 25B—Graphs showing that GalNAc siHOTAIR, delivered either via weekly injection or daily eye drops, reversed diabetes-induced upregulation of VEGFC (25A) and ANGPTL4 (25B) mRNAs in the retinas of diabetic rats. For injection treatment, diabetic mice were given 1 injection (10 μL) of GalNAc siHOTAIR and sacrificed one week following treatment. For eye drop treatment, diabetic rats were given 1 eye drop (5 μL) of GalNAc siHOTAIR per day for 1 week and then sacrificed. Retinal tissues were harvested and examined. No toxicity was noted. [lncRNA and mRNA expression data shown as ratios to the non-diabetic condition; data are normalized to [3-Actin mRNA and presented as mean+standard deviation; *=significantly different from all other groups.]

FIGS. 26A to 26B—Graphs showing that GalNAc siHOTAIR, delivered either via weekly injection or daily eye drops, prevented diabetes-induced upregulation of HOTAIR lncRNA (26A) and VEGFA mRNA (26B) in the retinas of diabetic mice. For injections, diabetic mice were given 1 injection (1 μL) of GalNAc siHOTAIR each week for 3 weeks and then sacrificed. For eye drops, diabetic mice were given 1 eye drop (2 μL) of GalNAc siHOTAIR per day for 3 weeks and then sacrificed. Retinal tissues were harvested and examined. No toxicity was noted. [lncRNA and mRNA expression data shown as ratios to the non-diabetic condition; data are normalized to [3-Actin mRNA and presented as mean+standard deviation; *=significantly different from all other groups.]

FIGS. 27A to 27B—Graphs showing that GalNAc siHOTAIR, delivered either via weekly injection or daily eye drops, prevented diabetes-induced upregulation of VEGFC (27A) and ANGPTL4 (27B) mRNAs in the retinas of diabetic mice. For injections, diabetic mice were given 1 injection (1 μL) of GalNAc siHOTAIR each week for 3 weeks and then sacrificed. For eye drops, diabetic mice were given 1 eye drop (2 μL) of GalNAc siHOTAIR per day for 3 weeks and then sacrificed. Retinal tissues were harvested and examined. No toxicity was noted. [lncRNA and mRNA expression data shown as ratios to the non-diabetic condition; data are normalized to [3-Actin mRNA and presented as mean+standard deviation; *=significantly different from all other groups, t=significantly different from the Diabetic group.]

FIGS. 28A to 28B—Graphs showing that GalNAc siHOTAIR, delivered either via weekly injection or daily eye drops, prevented diabetes-induced upregulation of HOTAIR lncRNA (28A) and VEGFA mRNA (28B) in the retinas of diabetic rats. For injections, diabetic rats were given 1 injection (10 μL) of GalNAc siHOTAIR each week for 3 weeks and then sacrificed. For eye drops, diabetic rats were given 1 eye drop (5 μL) of GalNAc siHOTAIR per day for 3 weeks and then sacrificed. Retinal tissues were harvested and examined. No toxicity was noted. [lncRNA and mRNA expression data shown as ratios to the non-diabetic condition; data are normalized to [3-Actin mRNA and presented as mean+standard deviation; *=significantly different from all other groups, t=significantly different from the Diabetic group.]

FIG. 29A to 29B—Graphs showing that GalNAc siHOTAI R, delivered either via weekly injection or daily eye drops, prevented diabetes-induced upregulation of VEGFC (29A) and ANGPTL4 (29B) mRNAs in the retinas of diabetic rats. For injections, diabetic rats were given 1 injection (10 μL) of GalNAc siHOTAIR each week for 3 weeks and then sacrificed. Foreye drops, diabetic rats were given 1 eye drop (5 μL) of GalNAc siHOTAIR per day for 3 weeks and then sacrificed. Retinal tissues were harvested and examined. No toxicity was noted. [lncRNA and mRNA expression data shown as ratios to the non-diabetic condition; data are normalized to [3-Actin mRNA and presented as mean+standard deviation; *=significantly different from all other groups.]

FIGS. 30A to 30B—30A: Graph illustrating high glucose-induced expression of asialoglycoprotein receptor (ASGR1) mRNA in human retinal endothelial cells treated to high (25 mM) glucose as compared with normal (5 mM) glucose. 30B: Graph illustrating increased expression of ASGR1 mRNA in the retina of diabetic animals as compared with non-diabetic controls. mRNA expressions are normalized to [3-Actin mRNA and presented as mean+standard deviation; *=significantly different from all other groups.

### DETAILED DISCLOSURE

### Definitions

In this specification and in the claims that follow, reference will be made to several terms that shall be defined to have the meanings below. All numerical designations, e.g., dimensions and weight, including ranges, are approximations that typically may be varied (+) or (−) by increments of 0.1, 1.0, or 10.0, as appropriate. All numerical designations may be understood as preceded by the term “about”.

The term “about,” particularly in reference to a given quantity, is meant to encompass deviations of plus or minus five percent.

As used in the specification and claims, the singular form “a”, “an” and “the” include plural references unless the context clearly dictates otherwise. For example, the term “a compound” includes a plurality of compounds, including mixtures thereof.

As used herein, the terms “comprising,” “including,” “having” are intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure. Embodiments defined by each of these transition terms are within the scope of this disclosure.

“Long non-coding RNAs (lncRNAs)” are a class of RNA transcripts larger than 200 base-pairs and possess limited protein-coding capacities. LncRNAs are dynamically regulated and present with distinct functionalities that facilitate chromatin remodelling and/or help govern the expression of genes involved in a multitude of biological and pathological processes, including development (26), cancer (27), and neurodegeneration (28).

“HOTAIR” (for HOX transcript antisense RNA) refers to a long non-coding (Inc) RNA transcript produced from a human gene located on chromosome 12 (spanning from 54,356,092 to 54,368,740 nucleotides (GRCh37/hg19)). Following transcription, splicing, and polyadenylation, HOTAIR transcripts do not encode for proteins, since these transcripts contain little or no open reading frames (GENBANK IDs: NR 047517.1, NR_003716.3, NR_047518.1).

MALAT1: metastasis-associated lung adenocarcinoma transcript 1 (MALAT1) is a highly conserved intergenic lncRNA that has implications in various cancers (29, 30), neurological disorders (31), and cardiovascular disease (32).

H19: a conserved and maternally imprinted lncRNA, is one of the earliest identified lncRNAs (33).

WISPER (Wisp2 super-enhancer-associated RNA) is a novel and recently identified lncRNA that plays a prominent role in regulating cardiac fibrosis after injury (34).

ZFAS1: Highly expressed in the heart, the lncRNA ZFAS1 (ZNFX Antisense RNA 1) is a regulator of organ development, cancer growth and metastasis, apoptosis, and cell cycle regulation (35, 36).

HULL: Highly upregulated in liver cancer (HULL) is a critical lncRNA that regulates angiogenesis, cell proliferation and migration, stem cell differentiation and lipid metabolism (37).

MIAT: Myocardial infarction-associated transcript (MIAT; also referred to as RNCR2, Gomafu, or AK028326) was originally identified in a case-control genome-wide association study, where 6 single nucleotide polymorphisms in the MIAT locus conferred susceptibility to myocardial infarction (MI) (38). Following this initial study, several experimental studies have emerged that shed light on the functional roles of MIAT in various biological and pathological processes, including schizophrenia (39), lung cancer (40), retinal and brain development (41, 42), and cataract formation (43).

ANRIL: Consisting of 19 exons and spanning nearly 126 kilobases (kb) (44), the antisense RNA to INK4 locus (ANRIL; also known as CDKN2B-AS1) gene gives rise to a 3.8-kb lncRNA that is prominently deregulated in cardiovascular disease (45) and several cancers (46).

The VEGF-A or VEGFA (Vascular endothelial growth factor A) gene is a member of the PDGFNEGF growth factor family. It encodes a heparin-binding protein, which exists as a disulfide-linked homodimer. This growth factor induces proliferation and migration of vascular endothelial cells and is essential for both physiological and pathological angiogenesis. Indeed, VEGF-A is known as an important contributor to the development of diabetic macular edema and proliferative DR. In this document and in the figures, “VEGF” is used to refer to VEGFA or VEGF-A.

ANGPTL4 refers to a gene that encodes for Angiopoietin-like 4 protein, which is implicated in the metastatic process by modulating vascular permeability, cancer cell motility and invasiveness. Additionally, ANGPTL4 is known to be a potent angiogenic mediator in proliferative DR.

VEGFC refers to Vascular endothelial growth factor C (VEGF-C), a member of the platelet-derived growth factor/vascular endothelial growth factor (PDGFNEGF) family. VEGFC acts on endothelial cells mainly through its receptor VEGFR-3. It promotes endothelial survival, growth, migration and angiogenesis. It also regulates vascular permeability. The effect on blood vessels is mediated via its primary receptor VEGFR-3 or its secondary receptor VEGFR-2. VEGFC is encoded in humans by the VEGFC gene, located on chromosome 4q34. In diabetic retinopathy, VEGF-C has been shown to be a major player in macular edema (Li Y, et al., Invest Ophthalmol Vis Sci. 2019 Oct. 1; 60(13):4084-4096. doi: 10.1167/iovs.19-26767). Currently used anti-VEGF therapies do not block VEGFC, and there are no known commercially available VEGFC blockers.

Ocular diseases that can be treated or diagnosed or prevented according to embodiments of the present disclosure include age-related macular degeneration (AMD), both dry AMD and wet AMD, and eye-related diabetes-induced conditions or diseases.

Eye-related diabetes-induced conditions or diseases that can be treated or diagnosed or prevented according to embodiments presented herein include, non-proliferative and proliferative diabetic retinopathy, neovascular glaucoma, ischemic retinopathy, and diabetic complications such as diabetic nephropathy, diabetic cardiomyopathy, and diabetic neuropathy.

The terms “siRNA” and “short interfering RNA” are interchangeable and refer to single-stranded or double-stranded RNA molecules that are capable of inducing RNA interference of a target mRNA molecule or a target lncRNA molecule. siRNA molecules typically have a duplex region that is between 18 and 30 base pairs in length. There may be many different versions of siRNAs that induce RNA interference of one target RNA molecule. For example, there are many HOTAIR siRNA molecules that interfere with HOTAIR lncRNA (see Tables 5 and 6). In this document, the general term “siRNA” includes any siRNA molecules capable of interfering with the target RNA molecule. For example, “siHOTAIR” refers to any siRNA molecule that interfere with HOTAIR lncRNA.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid molecule is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequence.

The term “complementary” and “complementarity” are interchangeable and refer to the ability of polynucleotides to form base pairs with one another. 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other and can be expressed as a percentage.

A “target site” or “target sequence” is the nucleic acid sequence recognized (i.e., sufficiently complementary for hybridization) by an antisense oligonucleotide or inhibitory RNA molecule.

The term “transfection” is used to refer to the uptake of foreign DNA or RNA by a cell. A cell has been “transfected” when exogenous DNA or RNA has been introduced inside the cell membrane. The term refers to both stable and transient uptake of the genetic material, and includes uptake, for example, of microRNA, siRNA, piRNA, lncRNA, or antisense nucleic acids. Transfection may be achieved with the aid of a transfection reagent or without a transfection reagent.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

An “inhibitor”, a term that in embodiments includes antagonists, is a molecule (e.g., microRNA, small interfering RNA (siRNA), PIWI-interacting RNA (piRNA), small nuclear RNA (snRNA), short hair-pin RNA (shRNA), antisense nucleic acid, ribozyme, or small molecule inhibitor) that inhibits, suppresses or causes the cessation of at least biological activity mediated by a gene, for example by interfering with the expression or transcription of the gene or by interfering with the interaction of the gene product to its target.

By a “HOTAIR inhibitor”, a term that includes antagonists of HOTAIR, is meant any molecule (e.g., microRNA, siRNA, piRNA, snRNA, antisense nucleic acid, ribozyme, or small molecule inhibitor) that inhibits, suppresses or causes the cessation of at least HOTAIR-mediated biological activity, for example by interfering with transcription of HOTAIR or interfering with the interaction of HOTAIR to its target, such as a Polycomb-group protein Polycomb Repressive Complex 2 (PRC2). In one embodiment, a HOTAIR inhibitor includes a GalNAc-modified siRNA (such as a GalNAc-modified HOTAIR siRNA) that reduces the amount and/or activity of HOTAIR by at least about 10% to about 100%, 20% to about 100%, 30% to about 100%, 40% to about 100%, 50% to about 100%, 60% to about 100%, 70% to about 100%, 10% to about 90%, 20% to about 85%, 40% to about 84%, 60% to about 90%, including any percent within these ranges, such as but not limited to 15%, 20%, 25%, 30%, 35%, 40%, 45%, 50%, 55%, 60%, 65%, 70%, 75%, 80%, 85%, 90%, 95%, 96%, 97%, 98%, and 99%.

An “effective amount” of a carbohydrate-modified nucleic acid molecule is an amount sufficient to effect beneficial or desired results. For example, in the case of GalNAc-siRNA and effective amount is an amount that inhibits, for example, the activity of the lncRNA HOTAIR, or an amount of a carbohydrate-modified nucleic acid molecule that inhibits the activity of VEGFA, VEGFC, ANGPTL4, miR-200b, miR-146a, ANRIL lncRNA, MALAT1 lncRNA, HULL lncRNA, H19 lncRNA, MIAT lncRNA and ZFAS1 lncRNA to name a few. An effective amount can be administered in one or more administrations, applications, or dosages.

By “anti-angiogenesis activity” is intended a reduction in the rate of branching, and hence a decline in angiogenesis. Such activity can be assessed using animal models.

The terms “subject,” “individual,” and “patient,” are used interchangeably herein and refer to any mammalian subject for whom diagnosis, prognosis, treatment, prevention or therapy is desired, particularly humans. Other subjects may include cattle, dogs, cats, guinea pigs, rabbits, rats, mice, horses, and so on. In some cases, the methods and compositions of the present disclosure find use in experimental animals, in veterinary application, and in the development of animal models for disease, including, but not limited to, rodents including mice, rats, and hamsters; primates, and transgenic animals.

As used herein, a “biological sample” refers to a sample of tissue or fluid isolated from a subject, including but not limited to, for example, vitreous humor (VH), tears, urine, blood, plasma, serum, fecal matter, bone marrow, bile, spinal fluid, lymph fluid, extracellular vesicles (e.g., exosomes), samples of the skin, external secretions of the skin, respiratory, intestinal, and genitourinary tracts, saliva, milk, blood cells, organs, biopsies, and also samples containing cells or tissues derived from the subject and grown in culture, and in vitro cell culture constituents, including but not limited to, conditioned media resulting from the growth of cells and tissues in culture, recombinant cells, stem cells, and cell components.

The terms “quantity,” “amount,” and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

“Administration” can be carried out in one dose, continuously or intermittently throughout the course of treatment. Methods of determining the most effective means and dosage of administration are known to those of skill in the art and will vary with the composition used for therapy, the purpose of the therapy, the target cell being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and target cell or tissue. Routes of administration include systemic administration and local administration. Systemic administration is a route of administration of medication, nutrition or other substance into the circulatory system so that the entire body is affected. Local administration is a route where the effect is generally focused to a site of action (the eye in this disclosure) thereby reducing risks of systemic side effects. Non-limiting examples of route of local administration include (i) topical administration, such as with the application of eyedrops or hydrogels and (ii) injection (subretinal, subconjunctival or intravitreal injections).

“Prevent” is used to mean obtaining a prophylactic effect in terms of completely or partially preventing frostbite injury from occurring.

As used herein, the terms “treating,” “treatment” and the like are used herein to mean a partial or complete cure of an eye disease or condition and/or adverse effect attributable to the eye disease or condition.

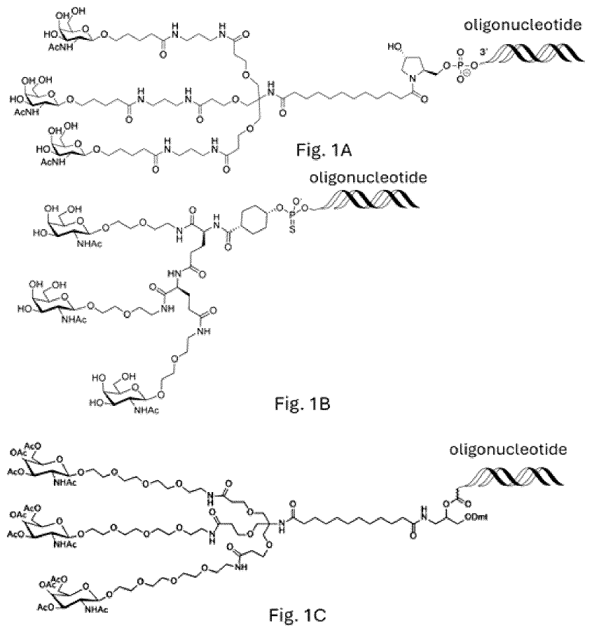

“Carbohydrate (CHO)” or “CHO conjugate” or “CHO moiety” are used in the present disclosure to refer to a sugar. In one embodiment the CHO is a monosaccharide. In another embodiment, the CHO is an acetylated amino sugar, including an acetylated amino monosaccharide. In one embodiment, the CHO is a multivalent galactose, such as GalNAc, GalNAc modification or a GalNAc derivative. In certain embodiments of the compositions and methods of the present disclosure, the CHO is one or more GalNAc moieties or GalNAc modifications or derivatives linked or attached, optionally through a monovalent, bivalent or trivalent branched linker, to the NA (see FIG. 1A-C, showing, as an example, the chemical structures of GalNAc moiety covalently bound to a NA, such as a siRNA. Each structure may include up to three GalNAc molecules attached through a trivalent branched linker to the NA. GalNAc derivatives/moieties are described, for example, in U.S. Pat. No. 8,106,022 and U.S. Pat. Appl. Publ. No. 20200270611, which are incorporated herein by reference. In this document, unless otherwise specified, “GalNAc” is used to refer to GalNAc, GalNAc modifications and GalNAc derivatives/moieties.

“Nucleic acid (NA) molecule” is used in this disclosure to refers to a single stranded or a double stranded nucleotide chain or oligonucleotide. In one embodiment, the NA is an inhibitor (as defined in this disclosure).

In this document, the terms “a CHO modified NA” and “a NA having a CHO” are used interchangeably and both terms are abbreviated “CHO-NA.”

### Overview

Regulatory lncRNAs are important in the pathogenesis and progression of ocular diseases such as diabetic retinopathy (DR) and age-related macular degeneration (AMD) (both dry AMD and wet AMD). Thus, the present disclosure relates to targeted silencing of pro-angiogenic lncRNAs such as HOTAIR Inc RNA, to treat and prevent ocular diseases such as DR, dry AMD and wet AMD.

The present disclosure relates to the use of nucleic acid molecules (NA) conjugated to, or linked to, or modified with, or having a carbohydrate (CHO) to treat or prevent eye related diseases or conditions, with the proviso that said CHO when linked to the NA treats or prevents an ocular condition. In one embodiment, said CHO linked to the NA prevents high-glucose-induced or diabetes-induced upregulation or downregulation of one or more agents known to induce an ocular disorder such as diabetic retinopathy (DR) and AMD. The present disclosure also relates to nucleic acid molecules having or modified with a CHO conjugate (CHO-NA) for use in treating or preventing eye related diseases or conditions. In one embodiment, the CHO conjugate or moiety is a monosaccharide. In another embodiment, the CHO conjugate is a multivalent galactose. In another embodiment, the CHO conjugate is GalNAc or a GalNAc derivative.

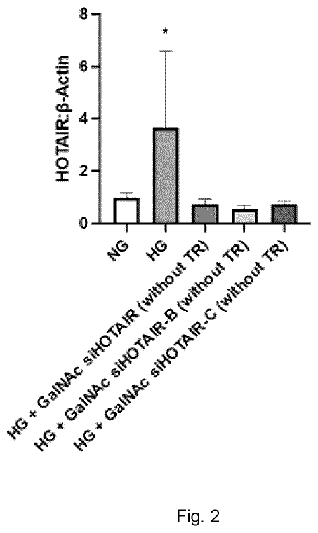

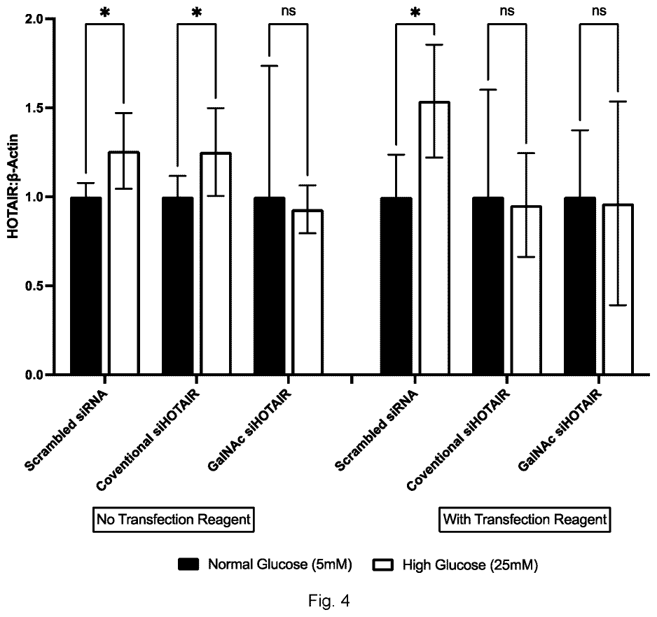

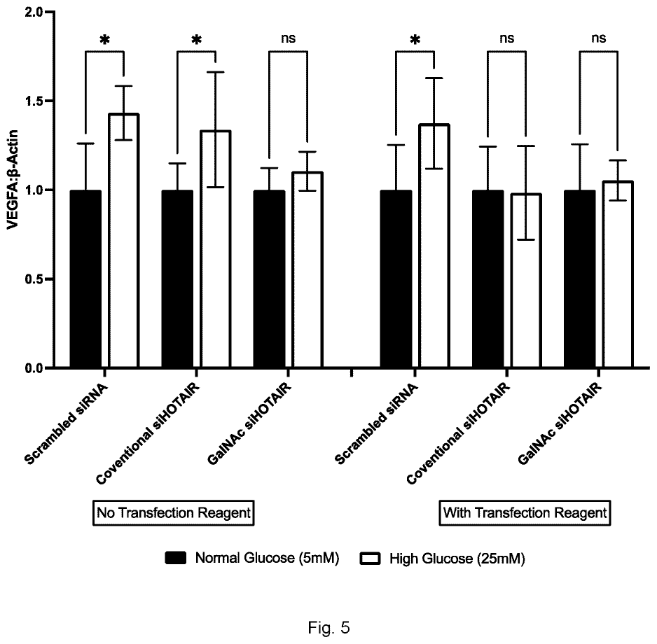

Unexpectedly, CHO-NA has been found to be effective both in treating and preventing high glucose-induced upregulation or downregulation of agents known to induce ocular disorders such as DR (see FIGS. 2-29). While unmodified therapeutic NAs (i.e., NAs not modified with CHO, also referred to as naked or conventional NA) such as siHOTAIR are also effective in preventing high glucose-induced upregulation or downregulation of agents known to induce ocular disorders such as DR, the naked NAs only work when administered in combination with a transfection agent (see FIGS. 4 and 5). On the other hand, the CHO-NAs of the present disclosure have been shown to treat and prevent high glucose-induced upregulation or downregulation of agents known to induce ocular disorders both with and without a transfection agent (see for example FIGS. 4 and 5 and FIGS. 20 to 29).

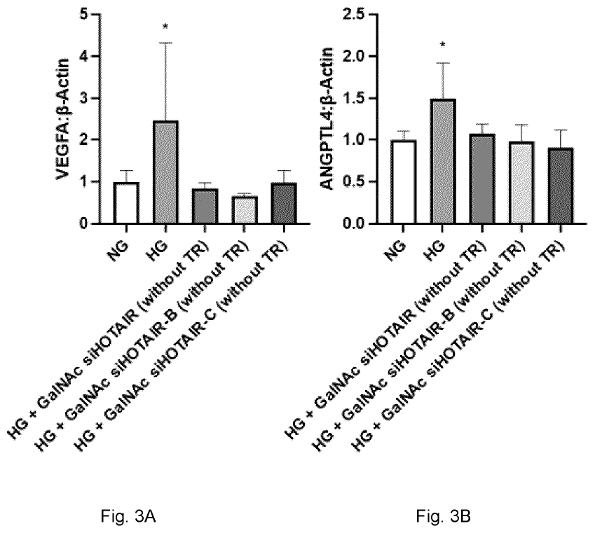

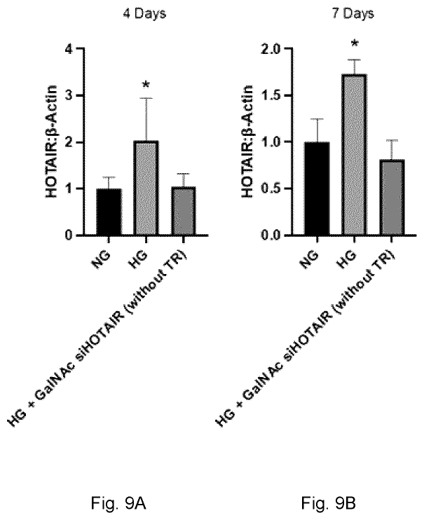

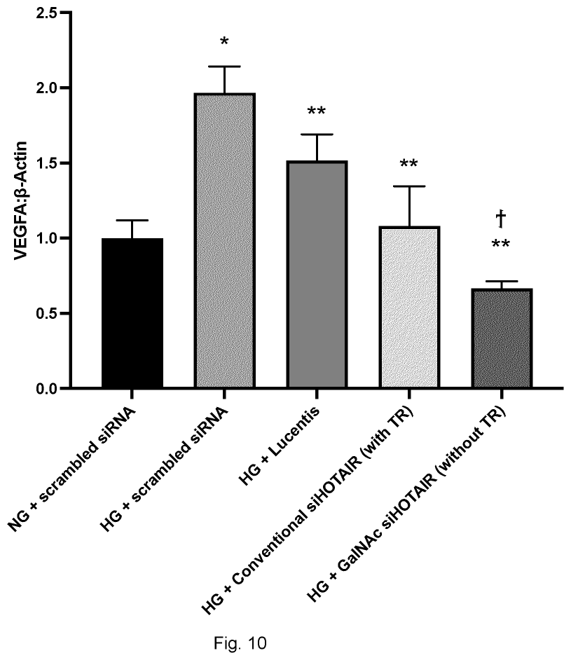

FIGS. 1A-1C illustrate the chemical structures of three different GalNAc-modified oligonucleotide molecules. When the oligonucleotide in the three molecules of FIGS. 1A-1C is siHOTAIR, all three molecules effectively target HOTAIR (see FIG. 2) and all three molecules affect downstream molecules such as VEGFA (FIG. 3A) and ANGPTL4 (FIG. 3B). All remaining data was performed using the structure of FIG. 1A, however, one of ordinary skill in the art understands that the structures of FIGS. 1B and 10 as well as any other structure of a CHO-NA may be used in the embodiments of the present disclosure.

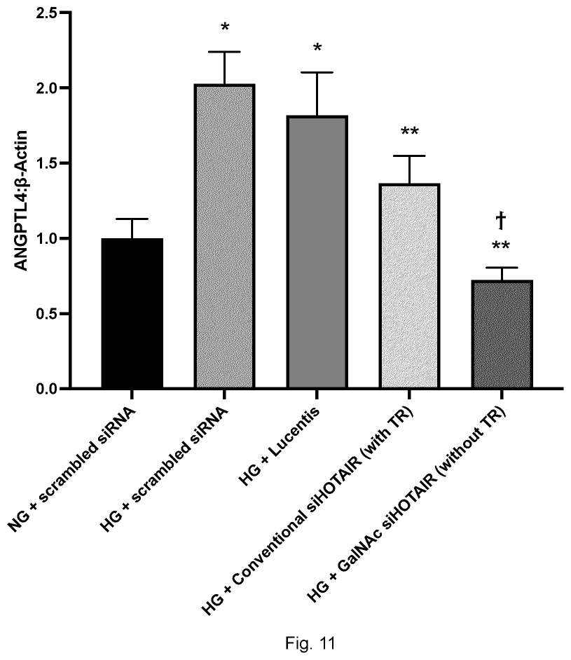

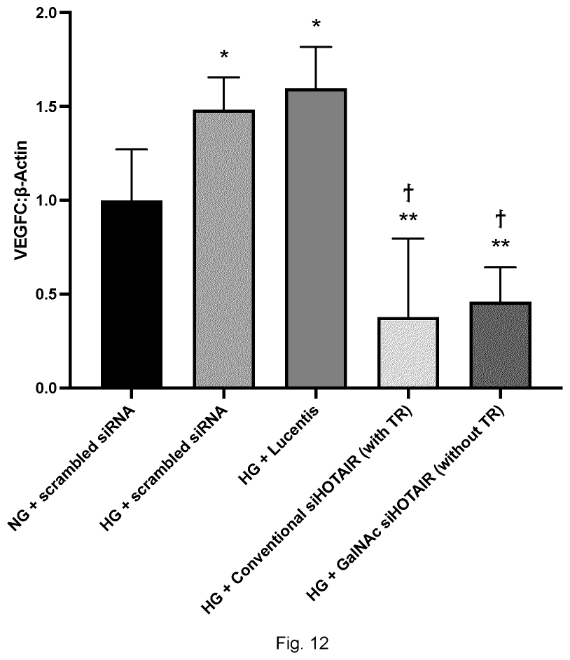

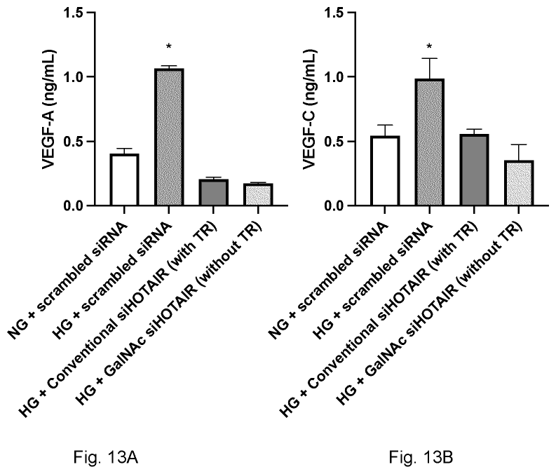

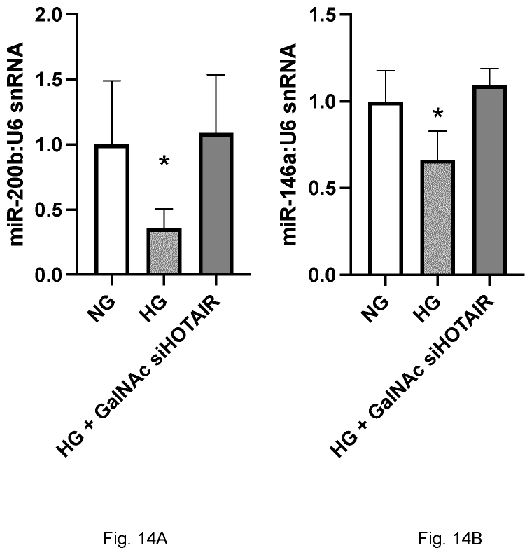

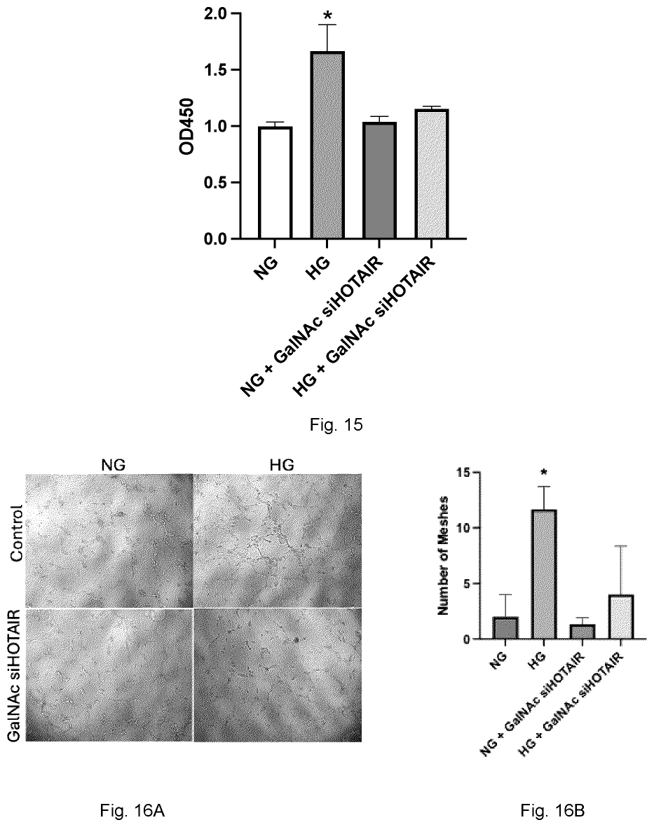

With reference to the figures, in one embodiment, GalNAc siHOTAIR is shown to prevent high glucose induced upregulation of HOTAIR (FIGS. 2, 4, 7, 9A-B), VGFA (FIGS. 3A, 5, 6, 8, 10), ANGPTL4 (FIGS. 3B and 11), VEGFC (FIGS. 12 and 13B) in vitro in human retinal endothelial cells (see FIGS. 4-10), to prevent high glucose mediated inhibition of miR-200b (FIG. 14A and of miR-146a (FIG. 14B), to prevent high glucose-induced increase in permeability in human retinal endothelial cells (FIG. 15).

In another embodiment, GalNAc siHOTAIR reduces diabetes induced upregulation of HOTAIR VGFA, ANGPTL4 and VEGFC in vivo in the retina of diabetic animals (see FIGS. 20A, 20B, 21A, 21B, 22A, 22B, 23A, 23B, 24A, 24B, 25A and 25B).

Unexpectedly, GalNAc siHOTAIR is shown to prevent high glucose induced upregulation of HOTAIR and VGFA when used with or even without a transfection reagent, while conventional siHOTAIR only prevents high glucose induced upregulation of HOTAIR and VGFA when used in combination with a transfection reagent (see FIGS. 4 and 5).

In another embodiment, GalNAc siHOTAIR prevents diabetes-induced upregulation of HOTAIR and VGFA, ANGPTL4 and VEGFC in vivo in the retina of diabetic animals (see FIGS. 26A, 26B, 27A, 27B, 28A, 28B, 29A and 29B).

Furthermore, eyedrops having GalNAc siHOTAIR reverses diabetes induced upregulation of HOTAIR and VGFA in the retina, while eyedrops having conventional siHOTAIR is unable to reduce diabetes-induced upregulation of HOTAIR, VGFA, ANGPTL4 and VEGFC in the retina (see FIGS. 19, 20A-B, 21A-B).

Given the unexpected findings of this disclosure (see FIGS. 2 to 29), the retina was then examined and found to express ASGR1, a receptor of galactose and GalNAc (see FIGS. 30A and 30B). Furthermore, it was found that the expression of ASGR1 increases under high glucose levels (FIG. 30A) and in the retina of a diabetic subject (FIG. 30B).

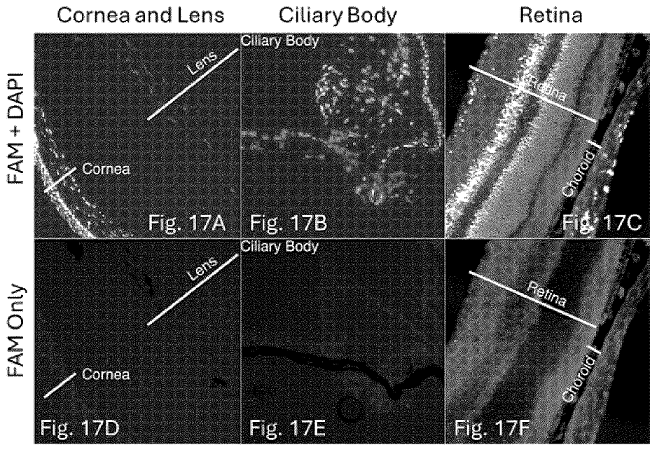

In one embodiment, the GalNAc-modified NA molecules of the present disclosure showed specific localization of the GalNAc-modified NA molecules to the retina (see FIGS. 17A-17F). As such, in another embodiment, the present disclosure provides for a composition comprising a CHO modified nucleic acid molecule (CHO-NA) that is useful to target retina tissue. In another embodiment, the present disclosure provides for a method to target or deliver a drug (i.e., any substance (other than food) that is used to prevent, diagnose, treat, or relieve symptoms of a disease or abnormal condition), such as a nucleic acid molecule, to the retina, the method comprising contacting the retina with the drug linked to a ligand of the ASGR. In one embodiment, the target or deliver is specific to the retina. In one embodiment, the ligand (artificial or naturally occurring molecule that binds to the ASGR) is a carbohydrate moiety (CHO). In one embodiment, the ligand is an acetylated amino sugar. In one embodiment, the ligand of the ASGR is a monosaccharide. In another embodiment, the ligand is a multivalent galactose. In another embodiment, the ligand is GalNAc or a GalNAc derivative or GalNAc modification (in this document the terms “GalNAc derivative” and “GalNAc modification” are used interchangeably).

In one embodiment, the nucleic acid molecule in the CHO-NA is a siRNA, a microRNA, a piRNA, a snRNA, a shRNA, an antisense nucleic acid or a ribozyme.

In one embodiment, the CHO-NA is a CHO modified siRNA (CHO-siRNA). In one embodiment, the siRNA is an inhibitor of a lncRNA. In one embodiment, the lncRNA is one or more of HOTAIR lncRNA, MALAT1 lncRNA, ANRIL lncRNA, WISPER lncRNA, ZFAS1 lncRNA, H19 lncRNA, MIAT lncRNA, HULL lncRNA. In another embodiment, the siRNA is HOTAIR siRNA (siHOTAIR), VEGFA siRNA (siVEGFA), MALAT1 siRNA (siMALAT1), ANRIL siRNA (siANRIL), WISPER siRNA (siWISPER), ZFAS1 siRNA (siZFAS1), H19 siRNA (siH19), MIAT siRNA (siMIAT), HULL siRNA (siHULC).

### Treatment/Prevention of Ocular Conditions

In one embodiment, the present disclosure relates to a method of treating or preventing an ocular condition or disease comprising, or alternatively consisting of, local administration to an eye (one eye or both eyes) of a subject having or suspected of having the ocular condition or disease (may also be referred to as a subject in need), a CHO modified nucleic acid molecule (CHO-NA) effective for the treatment or prevention of the ocular condition or disease. In one embodiment, the nucleic acid molecule in the CHO-NA is a siRNA, a microRNA, a piRNA, a snRNA, a shRNA, an antisense nucleic acid or a ribozyme. In embodiments of the present disclosure, the CHO-NA is a CHO-siRNA, e.g. GalNAc-siRNA or GalNAc derivative-siRNA. CHO-siRNAs of the present disclosure include one or more of CHO-siHOTAIR, CHO-siANRI L, CHO-siMALAT1, CHO-siWISPER, CHO-siH19, CHO-siMIAT, CHO-siHULC and/or CHO-siZFAS1. In embodiments, the CHO-NA is provided in a therapeutically effective amount. In one embodiment, the method of treating or preventing an ocular condition or disease of the present disclosure includes, or alternatively consists of, the administration of the CHO-NA in combination with a transfection agent. In another embodiment, the method of treating or preventing an ocular condition or disease of the present disclosure is devoid of, or excludes, a transfection agent (i.e., a transfection agent is not used in the method of treating or preventing the ocular condition). In embodiments, the CHO-NA is administered in combination with (i) another different CHO-NA, (ii) in combination with another agent known to treat or prevent the ocular disease, or (iii) in combination with both (i) and (ii).

In one embodiment, the present disclosure relates to a nucleic acid molecule having a carbohydrate moiety (CHO) (CHO-NA) for use in the treatment or prevention of an ocular condition or disease in a subject having or suspected of having the ocular condition or disease. In one embodiment, the nucleic acid molecule in the CHO-NA is a siRNA, a microRNA, a piRNA, a snRNA, a shRNA, an antisense nucleic acid or a ribozyme. In one embodiment the CHO-NA is used for local administration to an eye of the subject. In one embodiment the CHO-NA is formulated for local administration to an eye of the subject. In embodiments of the present disclosure, the CHO-NA is a CHO-siRNA. In one embodiment, CHO-siRNAs include one or more of GalNAc-siHOTAIR, GalNAc-siANRIL, GalNAc-siMALAT1, GalNAc-siWISPER, CHO-siH19, CHO-siMIAT, and/or GalNAc-siZFAS1. In embodiments, the GalNAc-NA is provided in a therapeutically effective amount. In one embodiment, the GalNAc-NA is used in combination with a transfection agent. In one embodiment, the GalNAc-NA is used free of a transfection agent, i.e., the GalNAc-NA is used without a transfection agent.

In another embodiment, the present disclosure relates to a GalNAc modified NA (GalNAc-NA) for use in the manufacture of a medicament for the treatment or prevention of an ocular condition or disease in a subject having or suspected of having the ocular condition or disease. In one embodiment the medicament is used for local administration to an eye of the subject. In one embodiment the medicament is formulated for local administration to an eye of the subject. In embodiments of the present disclosure, the GalNAc-NA in the medicament is provided in a therapeutically effective amount. In one embodiment, the medicament includes a transfection agent. In another embodiment, the medicament is devoid of, is free of, a transfection agent. In one embodiment, the nucleic acid molecule in the GalNAc-NA is a siRNA, a microRNA, a piRNA, a snRNA, a shRNA, an antisense nucleic acid or a ribozyme. In one embodiment, the GalNAc-NA is a GalNAc-siRNA. In one embodiment, GalNAc-siRNAs include one or more of GalNAc-siHOTAIR, GalNAc-siANRIL, GalNAc-siMALAT1, GalNAc-siWISPER, CHO-siH19, CHO-siMIAT, and/or GalNAc-siZFAS1.

In embodiments of the compositions, medicaments, methods, products for use and uses of the present disclosure, the CHO or CHO conjugate is a monosaccharide that when linked to a therapeutic NA serves as an inhibitor of at least one biological activity in the eye associated with the ocular disease. In another embodiment, the CHO or CHO conjugate is a multivalent galactose that is a ligand to a GalNAc receptor in the retina. In another embodiment, the CHO or CHO conjugate is GalNAc or a GalNAc derivative that is a ligand to a GalNAc receptor in the retina. In the case of CHO being GalNAc, the abbreviations are made by replacing “CHO” with “GalNAc”, e.g. GalNAc-siRNA.

In embodiments of the compositions, medicaments, methods, products for use and uses of the present disclosure, the GalNAc-siRNA of the present disclosure is a GalNAc-modified HOTAIR siRNA. In embodiments, the HOTAIR siRNA (also referred to a siHOTAIR) in the GalNAc-modified HOTAIR siRNA comprises a sequence selected from SEQ ID NO: 22, SEQ ID NO: 24, SEQ ID NO: 26, and SEQ ID NO:28. In embodiments, the siHOTAIR comprises a sequence selected from SEQ ID NO: 21, SEQ ID NO: 23, SEQ ID NO: 25, and SEQ ID NO:27. In one embodiment, the siHOTAIR hybridizes to a target sequence of HOTAIR long non-coding RNA (lncRNA) selected from SEQ ID NOs: 29-32, 41-44.

In embodiments, the HOTAIR siRNA in the GalNAc-modified HOTAIR siRNA is a double stranded RNA (dsRNA) that comprises an antisense strand that comprises, or consists essentially of, or consists of, an antisense sequence selected from SEQ ID NO: 22, SEQ ID NO: 24, SEQ ID NO: 26, and SEQ ID NO:28.

In embodiments, the HOTAIR siRNA in the GalNAc-modified HOTAIR siRNA is a dsRNA that comprises a sense strand that comprises, or consists of, a sense sequence selected from SEQ ID NO: 21, SEQ ID NO: 23, SEQ ID NO: 25, and SEQ ID NO:27.

In one embodiment, the local administration is one or more of subconjunctival injection or intravitreal injection or subretinal injection or topical administration (eye drops or hydrogel), or any other form of local administration. In another embodiment, the local administration (subretinal injection, subconjunctival injection or intravitreal injection, topical administration and so forth) of the CHO-NA (such as GalNAc-siRNA) is free of (i.e., devoid of, without, excludes) a transfection agent.

Topical administration includes application of eye drops, topical application with hydrogels (natural or synthetic) such as hydrogels in the form of contact lenses or in situ gels applied as solutions and so forth.

In one embodiment of the present disclosure, the ocular condition or disease that can be treated or prevented with the methods, uses, compositions and medicaments of the present disclosure is a condition or disease associated with an overexpression of the long non-coding RNA HOTAIR.

In embodiments, the ocular related diseases or conditions (including diseases or conditions associated with overexpression of the long non-coding RNA HOTAIR) is a diabetes-induced disease or condition. In embodiments, the ocular disease or condition associated with overexpression of the long non-coding RNA HOTAIR is one or more of: diabetic retinopathy (including non-proliferative and proliferative diabetic retinopathy and macular edema), diabetic nephropathy, diabetic cardiomyopathy, diabetic neuropathy, proliferative vitreoretinopathies, neovascular glaucoma, ischemic retinopathy, retinopathy secondary to retinal vein occlusion, as well as age-related macular degeneration (dry AMD and wet AMD), keloid formation, and wound healing. In embodiments, the disease or condition associated with overexpression of the long non-coding RNA HOTAIR is a condition in which anti-VEGF therapy is ineffective.

In embodiments, the present disclosure includes a method of modulating the expression of HOTAIR targets, the method comprising administering into a cell a CHO-siHOTAIR. In one embodiment, the expression of VEGFA, VEGF C/B, ANGPTL4, is reduced in the cell following administration of the CHO-siHOTAIR (e.g. GalNAc-siHOTAIR), in another embodiment, expression of miR-200b and miR-146a is increased following administration of CHO siHOTAIR (e.g. GalNAc-siHOTAIR).

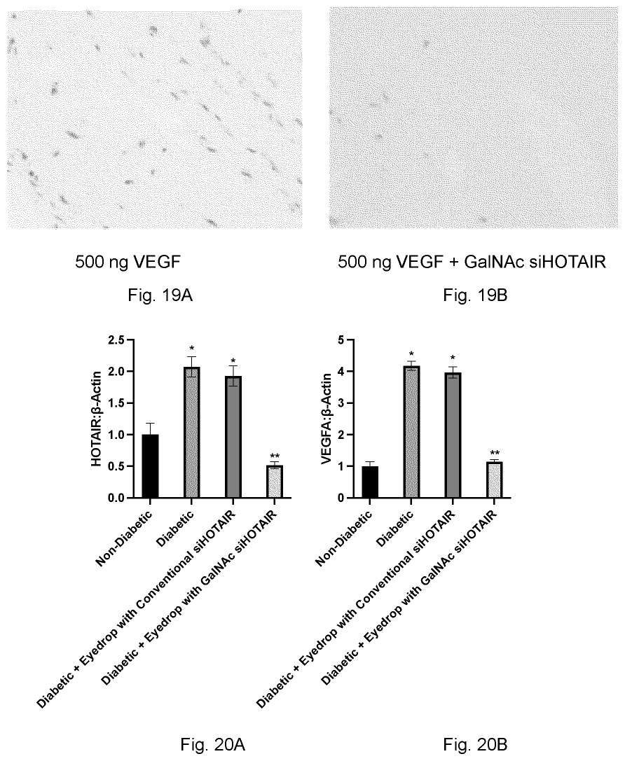

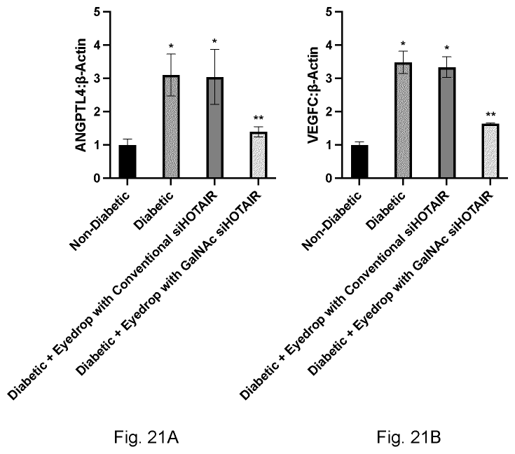

In one embodiment, the present disclosure shows that eyedrops comprising GalNAc siHOTAIR both reversed and prevented diabetes-induced upregulation of HOTAIR lncRNA (FIGS. 20A, 22A, 24A, 26A and 28A), VEGFA mRNA (FIGS. 20B, 22B, 24B, 26B and 28B), ANGPTL4 mRNA (FIGS. 21A, 23B, 25B, 27B and 29B) and VEGFC mRNA (FIGS. 21B, 23A, 25A, 27A and 29A), whereas conventional siHOTAIR failed to reverse diabetes-induced upregulation of HOTAIR lncRNA, VEGFA, VEGFC and ANGPTL4 (FIGS. 20A-B and 21A-B).

In another embodiment, the present disclosure shows that injectable compositions for the eye comprising GalNAc siHOTAIR both reversed and prevented diabetes-induced upregulation of HOTAIR lncRNA (FIGS. 22A, 24A, 26A and 28A), VEGFA mRNA (FIGS. 22B, 24B, 26B and 28B), ANGPTL4 mRNA (FIGS. 23B, 25B, 27B and 29B) and VEGFC mRNA (FIGS. 23A, 25A, 27A and 29A).

### Compositions

In one embodiment, the present disclosure provides for a topical composition comprising a GalNAc modified NA, such as GalNAc-siRNA, GalNAc microRNA, GalNAc-piRNA, GalNAc-snRNA, GalNAc-shRNA, GalNAc-antisense nucleic acid or GalNAc-ribozyme. In embodiments, the topical composition of the present disclosure is formulated as eye drops or hydrogel.

In one embodiment, the GalNAc modified NA in the topical composition is GalNAc modified siHOTAIR, GalNAc-siMALAT1, GalNAc-siANRIL, GalNAc-siWISPER and/or GalNAc-siZFAS1.

In one embodiment, the topical composition of the present disclosure is formulated as eyedrops, or as hydrogels (natural or synthetic) such as hydrogels in the form of contact lenses or in situ gels applied as solutions and so forth. In another embodiment, the topical composition of the present disclosure is formulated as an injectable composition for local administration (subretinal injection, subconjunctival injection or intravitreal injection, topical administration and so forth).

In one embodiment, the composition of the present disclosure includes a transfection reagent. In another embodiment, the topical composition of the present disclosure excludes, is free of, or devoid of, a transfection reagent.

In general, eye drops may be prepared in one of two ways: Dissolving a powder form of the active pharmaceutical ingredient (in this case the GalNAc-NA) and/or a preservative (in powder form) in a suitable vehicle (an aqueous component such as sterile water or a buffer solution), or by diluting a concentrated solution of the active pharmaceutical ingredient (i.e. the GalNAc-NA) using an aqueous component such as sterile water or suitable buffer solution.

Injections comprised GalNAc siHOTAIR dissolved in triple-distilled water at a concentration of 100 μM.

In addition to GalNAc-NA, the compositions of the present disclosure optionally include one or more pharmaceutically acceptable excipients or carriers.

In order to aid in the understanding and preparation of the present disclosure, the following illustrative, non-limiting examples are provided.

### EXAMPLES

### Example 1

### Methods

### Cell Culture

Human retinal microvascular endothelial cells (HRECs, Cell Systems, Kirkland, WA, USA; catalog number ACBRI 181) were cultured in endothelial basal media-2 (EBM-2, Lonza, Walkersville, MD, USA) containing endothelial growth media-2 (EGM-2) SingleQuots (Lonza). All cells were grown in 75 cm² culture flasks and maintained in a humidified incubator containing 5% C02 at 37° C. As described previously (1, 2, 3, 4), in order to reduce variability for experimentation, cells were used between passages three and six and the cellular densities were determined accordingly based on the type of culture plates used for each experiment. Generally, once 80% confluence was obtained post-seeding, ECs were cultured in serum and growth factor-free medium overnight before exposure to different D-glucose levels (final glucose concentrations of 5 mmol/L, mimicking normoglycemia [NG], and 25 mmol/L, mimicking hyperglycemia [HG]) for various durations; the selected glucose levels are based on a large volume of previous experiments (1, 2, 3, 4, 5, 6, 7). All in vitro or ex vivo experiments were independently repeated at least three times and performed with six replicates, unless specified. Lucentis® was added to the media at the same time high glucose was added.

siRNA Transfections

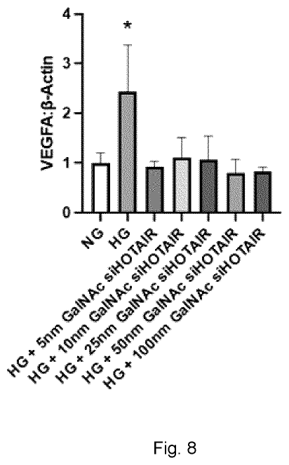

HRECs were transfected using 100 nM of scrambled siRNAs (ID number: AM4635 [SCR], Thermo Fisher Scientific) or pre-designed siRNAs targeting human HOTAIR and mouse HOTAIR as designed by us (Tables 1 and 2) using and without using transfection agent Lipofectamine 2000 (Invitrogen, Burlington, ON, Canada) and Opti-MEM reduced serum media (Thermo Fisher Scientific), as documented previously by us (1, 2, 3, 4, 6, 7). For GalNAc modified siRNAs, transfection reagent lipofectamine was used and was not used. Except for the experiment depicted in FIGS. 7 and 8, cells were transfected with 100 nM of each siRNA (GalNAc-modified siRNA and naked siRNA) for 3-4 hours and subsequently recovered in complete EBM-2 overnight. For the experiment depicted in FIGS. 7 and 8, cells were transfected with 5 nM, 10 nM, 25 nM, 50 nM and 100 nM of each siRNA. Cells were then serum starved the following morning, between 18-24 hours, and then incubated with specific glucose concentrations (5 mmol/L or 25 mmol/L) for 48 hours. The knockdown of the target genes was then confirmed using RT-qPCR. GalNAc modified siRNAs were prepared by and obtained from BocSciences.

### Diabetic Animal Models

The Western University Council for Animal Care Committee approved all animal models used in this study and experiments were performed in accordance with The Guide for the Care and Use of Laboratory Animals (NIH Publication 85-23, revised in 1996). Beginning with our initial two-month in vivo model male mice (C57/BL6 background; −25 g, 8 weeks old) were obtained (Charles River, Wilmington, MA, USA) and randomly divided into control and diabetic groups. Streptozotocin (STZ) was used to generate a type 1 diabetic animal model and methods of diabetes induction and monitoring have been previously described (1, 4, 5, 7). At two months following diabetes induction, animals were euthanized (n=10 for each treatment group) and retinal tissues were collected and used in part for RNA extraction and HOTAIR levels were assessed using RT-qPCR. The remaining retinal tissues were fixed in formalin and embedded in paraffin using standard methodology (1, 2, 4, 5).

### Mouse Eye Drops

Short-term eye drop treatments began 3 weeks post model induction (this is referred to as the “treatment” group). Diabetic mice (n=8) were picked up from the cage, scruffed securely, then held against a flat surface. 2 μL of GalNAc siHOTAIR [100 μM] was applied to the right eye using a micropipette. Mice were kept steady for 15 seconds following eye drop to ensure proper coverage of the eye. No anesthesia was applied, and no recovery period was needed. For the “treatment” group, eye drops were applied twice per day for 5 consecutive days. Retinal tissues were harvested on 5th day and examined. Total diabetic duration at time of harvesting was 1 month. No toxicity was noted.

Long-term eye drop treatments began 1 week post model induction (this is referred to as the “prevention” group). Diabetic mice (n=8) were given eye drops as described above. Eye drops were applied 5 days a week for 3 consecutive weeks. Retinal tissues were harvested at the end of the 3^{rd} week and examined. Total diabetic duration at time of harvesting was 1 month. No toxicity was noted.

### Mouse Intravitreal Injection

Short-term intravitreal injections began 3 weeks post model induction (this is referred to as the “treatment” group). For this, diabetic mice (n=8) were anesthetized using isoflurane in oxygen (induction at 4% isoflurane, maintenance at 2%; 1.5% O2 throughout). 1 μL of GalNAC siHOTAIR [100 μM] was injected into the right eyes of the mice using a 10 μL glass syringe (Hamilton, Reno USA) and a 34-gauge needle. Surgical positioning of the needle and the general duration of each intravitreal injection have been described previously (1,8). Left eyes received mock treatments of only water. Following injection, mice were kept under anesthesia for 1 extra minute, then moved to a heated surface to recover until normal ambulation was observed. Mice were sacrificed 1 week after the injection, and retinal tissues were harvested and examined. Total diabetic duration at time of harvesting was 1 month. No post-surgical ocular complications or toxicity was observed.

Long-term intravitreal injections began 1 week post model induction (this is referred to as the “prevention” group). For this, diabetic mice (n=8) were given intravitreal injections as described above, for 3 weeks. Mice were sacrificed at the end of the 3^{rd} week. Total diabetic duration at time of harvesting was 1 month. No post-surgical ocular complications or toxicity was observed.

FAM-labeled GalNAC siHOTAIR was synthesized by BocSciences and used to track the localization of GalNAc siHOTAIR. FAM-labeled GalNAc siHOTAIR was used in the same way as GalNAC siHOTAIR as described above. After 1 week of treatment, the mice were sacrificed, the eyes were harvested and mounted in optimal cutting temperature compound, then flash frozen in liquid nitrogen. 5 μm sections of the retina were cryosectioned and stained using DAPI (Abcam). Fluorescent images were taken using an epifluorescent microscope using a 10× objective lens.

### Rat Eye Drops

Short-term eye drop treatments began 3 weeks post model induction (this is referred to as the “treatment” group). Diabetic rats (n=6) were picked up from the cage, restrained with a towel, then held against a flat surface. 5 μL of GalNAc siHOTAIR [100 μM] was applied to the right eye using a micropipette. Rats were kept steady for 15 seconds following eye drop to ensure proper coverage of the eye. No anesthesia was applied, and no recovery period was needed. For the “treatment” group, eye drops were applied once per day for 5 consecutive days. Retinal tissues were harvested on 5th day and examined. Total diabetic duration at time of harvesting was 1 month. No toxicity was noted.

Long-term eye drop treatments began 1 week post model induction (this is referred to as the “prevention” group). Diabetic rats (n=6) were given eye drops as described above. Eye drops were applied 5 days a week for 3 consecutive weeks. Retinal tissues were harvested at the end of the 3^{rd} week and examined. Total diabetic duration at time of harvesting was 1 month. No toxicity was noted.

### Rat Intravitreal Injection

Short-term intravitreal injections began 3 weeks post model induction (this is referred to as the “treatment” group). For this, diabetic rats (n=6) were anesthetized using isoflurane in oxygen (induction at 4% isoflurane, maintenance at 2%; 1.5% 02 throughout). 10 μL of GalNAC siHOTAIR [100 μM] was injected into the right eyes of the rats using a 0.3 mL insulin syringe (Sol M, Chicago USA) and a 31-gauge needle. Surgical positioning of the needle and the general duration of each intravitreal injection have been described previously (1,8). Left eyes received mock treatments of only water. Following injection, rats were kept under anesthesia for 1 extra minute, then moved to a heated surface to recover until normal ambulation was observed. Rats were sacrificed 1 week after the injection, and retinal tissues were harvested and examined. Total diabetic duration at time of harvesting was 1 month. No post-surgical ocular complications or toxicity was observed.

Long-term intravitreal injections began 1 week post model induction (this is referred to as the “prevention” group). For this, diabetic rats (n=6) were given intravitreal injections as described above, for 3 weeks. Rats were sacrificed at the end of the 3^{rd} week. Total diabetic duration at time of harvesting was 1 month. No post-surgical ocular complications or toxicity was observed.

### RNA Isolation and Quantitative Real-Time Polymerase Chain Reaction (RT-qPCR)

As extensively described by us (1-7), total RNA was extracted using the TRIzol reagent (Invitrogen). Once total RNA was obtained, a spectrophotometer (260 nm; Gene Quant, Pharmacia Biotech, USA) was used to quantify RNA concentrations in which 1-2 μg of total RNA was reverse transcribed to complementary DNA (cDNA) using a high-capacity cDNA reverse-transcription kit (Applied Biosystems/Thermo Fisher Scientific). cDNA was then amplified in the LightCycler 96 System (Roche Diagnostics, Laval, QC, CAN) using the SYBR-green master mix (Takara Bio, Mountain View, CA, USA) and specific primers for the genes of interest (Sigma; Tables 1-2). RT-qPCR results were analyzed using the LightCycler 96 SW 1.1 software (Roche) and expression levels were calculated by the relative standard curve method using [3-actin as an internal control for sample normalization.

### Protein Quantification Using ELISA

Total protein was isolated using RI PA buffer (Millipore) and quantified using the BCA assay (Thermo Fisher Scientific) according to the manufacturer's instructions. ELISA kits for human Vegf-A and Vegf-C ELISA kits (Biomatik) were purchased and ELISA was performed according to the manufacturer's instructions.

### In Vitro Tube Formation Assay

Human retinal endothelial cells were seeded in 96-well plates coated with phenol red-free, reduced growth factor basement membrane extract after transfection as described previously. Full growth medium (EBM-2 with EGM-2) was used during seeding. 1 hour after cells have seeded, full growth medium was replaced with low serum medium containing low (5 mM) or high (25 mM) concentrations of glucose. Cells were incubated for 6 hours and images were taken to assess the progress tube formation (angiogenesis). Analysis of tube formation was done using ImageJ software.

In vitro Endothelial Transwell Permeability Assay

Human retinal endothelial cells were seeded on a transwell insert membrane endothelial growth medium (EBM-2 with EGM-2). The inserts were then transferred to wells containing the same growth medium and incubated until fully confluent. Cells were then treated to low serum medium (300 μL) containing either low (5 mM) or high (25 mM) concentrations of glucose. After 48 hours of glucose treatment, 5 μL streptavidin-horse radish peroxidase was added to the insert. The inserts were then transferred to new wells containing 1 mL low serum medium and incubated for 24 hours. After incubation, 20 μL of medium from the wells were transferred into a 96-well plate and 50 μL of TMB substrate was added. The plate was shaken until color change occurred, at which time 50 μL of stop solution was added and the optical density at 450 nm was read using an ELISA plate reader. High OD450 was indicative of more enzyme leaking into the low serum medium, and therefore higher permeability.

### In Vivo Angiogenesis Assay Using Matrigel Plug:

We used Matrigel plug assay for in vivo evaluation of angiogenesis and efficacy of our siRNAs. Growth factor reduced Matrigel supplemented with hVEGF (500 ng/plug) were injected into the backs of adult mice. Matrigel plugs with no-siRNA and plugs with VEGF blockers were used as controls. The following groups were used:
a) VEGFA (VEGF) (500 ng)
b) VEGF+GalNAc-siHOTAIR

The resulting angiogenesis were evaluated after 14 days following injection of the Matrigel plug, and histological examinations using H&E and CD31 immunostaining. Matrigel area (μm²), endothelial cell areas (μm²), endothelial cell ingrowth (%), and maximal endothelial cell depth (μm) were measured for quantification of angiogenesis using Image J software (10,11).

### Statistical Analyses

Statistical differences were evaluated between groups using GraphPad Prism 7 (La Jolla, CA, USA). Data were considered statistically significant if the P value was less than 0.05. All quantitative data for the in vitro experiments are presented as mean± SEM, while all in vivo data are presented as mean±SD. Experiments were performed in triplicate (n=6 per group), unless specified. Statistical significance for samples with non-parametric distribution was identified using the Mann-Whitney U test, while two-tailed Student's t-test (when comparing two conditions) or one-way ANOVA (for multiple comparisons; followed by Tukey's post hoc test) was applied for parametric variables.

TABLE 1
qPCR primers for human-specific genes

| Target Gene (Human): | Oligonucleotide Sequence (5′43′): | SEQ ID: |
|---|---|---|
| ACTB | F: TGTGGATCAGCAAGCAGGAG | 1 |
| | R: TGCGCAAGTTAGGTTTTGTC | 2 |
| HOTAIR | F: GGTAGAAAAAGCAACCACGAAGC | 3 |
| | R: ACATAAACCTCTGTCTGTGAGTGCC | 4 |
| VEGFA(VEGF) | F: GAACTTTCTGCTGTCTTGGG | 5 |
| | R: CTTCGTGATGATTCTGCCCT | 6 |
| VEGFC | F: CTCTCTCTCAAGGCCCCAAA | 7 |
| | R: AGACTTGGGCCTCTGTTACC | 8 |
| ANGPTL4 | F: GGACACGGCCTATAGCCTG | 9 |
| | R: CTCTTGGCGCAGTTCTTGTC | 10 |

TABLE 2
qPCR primers for mouse-specific genes

| Target Gene | Oligonucleotide Sequence (5′43′) | SEQ ID: |
|---|---|---|
| Actb | F: TGTGGATCAGCAAGCAGGAG | 11 |
| | R: TGCGCAAGTTAGGTTTTGTC | 12 |
| Hotair | F: GCGCCAACGTAGACCAAAAG | 13 |
| | R: TCTACCGATGTTGGGGACCT | 14 |
| Vegfa(Vegfi | F: ATGCGGATCAAACCTCACCA | 15 |
| | R: CTTTCTTTGGTCTGCATTCAC | 16 |
| Vegfc | F: CGCTGTGTCCCATCGTATTG | 17 |
| | R: AGACTTGGGCCTCTGTTACC | 18 |
| Angptl4 | F: TTGGTACCTGTAGCCATTCC | 19 |
| | R: GAGGCTAAGAGGCTGCTGTA | 20 |

### Results

### Overview for Cell Results in Cell

### 1. Introduction of 3 GalNAc-Modified NA Molecules (See FIGS. 1A-1C)

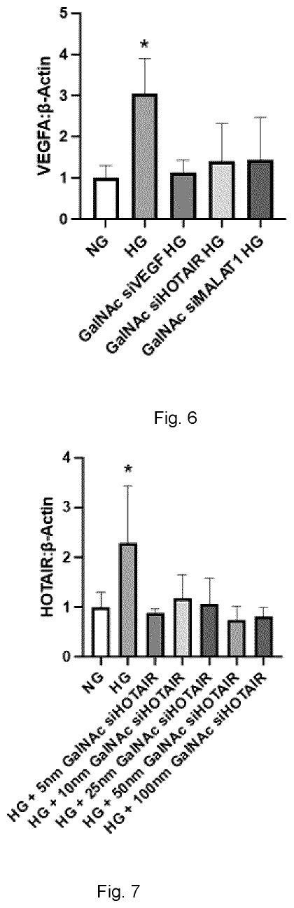

(a) When the NA is siHOTAIR, all 3 GalNAc-modified siHOTAIR effectively target HOTAIR (FIG. 2).
(b) When the NA is siHOTAIR, all 3 GalNAc-modified siHOTAIR affect downstream molecules such as VEGFA and ANGPTL4 (see FIGS. 3A-3B).
   2. Biochemical Effects of GalNAc Modified siHOTAIR:
(a) works without transfection reagent (FIGS. 4, 5, 10, 11, 12, 13A, 13B); (b) has a similar efficacy at VEGFA inhibition compared to GalNAc-modified siVEGFA (FIG. 6); (c) is effective at low dosages (FIGS. 7 and 8); (d) is effective at inhibiting both HOTAIR (FIGS. 2, 4, 7, 9A, 9B, and VEGFA (FIGS. 3A, 5, 6, 8, 10, 13A); (e) has long duration of efficacy (FIGS. 9A and 9B); (f) targets more angiogenic mediators compared to Lucentis (FIGS. 10, 11 and 12); (g) also targets pathogenic miRNAs (FIGS. 14A-14B).
   3. Functional Effects of GalNAc siHOTAIR
(a) prevents HG-induced increase in permeability (FIG. 15); (b) prevents HG-induced increase in tube formation/angiogenesis (FIGS. 16A and 16B).
   Three Different GalNAC-Modified siHOTAIRs are Effective at Preventing High Glucose-Mediated Induction of Angiogenic Factors

Three GalNAC-modified siHOTAIRs referred to a GalNAc siHOTAIR, GalNAc siHOTAIR-B and GalNAc-siHOTAIR-C, based, respectively, on the structures depicted in FIGS. 1A, 1B and 10 were shown to prevent high glucose-mediated induction of angiogenic factors HOTAIR lncRNA, VEGFA and ANGPTL4. Cells cultured under high glucose conditions showed significant glucose-induced upregulation of HOTAIR lncRNA (FIG. 2), VEGFA (FIG. 3A) and ANGPTL4 (FIG. 3B), without any intervention. Treatment with each of the three GalNAC-modified siHOTAIRs (GalNAc siHOTAIR, GalNAc siHOTAIR-B, or GalNAc siHOTAIR-C) reduced expression of HOTAIR lncRNA (FIG. 2), VEGFA (FIG. 3A) and ANGPTL4 (FIG. 3B) in cells cultured in high glucose conditions. In view these results, the reminder of the experiments were performed using GalNAc siHOTAIR (FIG. 1A).

### Human Retinal Endothelial Cell (HREC) Culture

Unmodified siHOTAIR was transfected into human retinal endothelial cell (HREC) culture with and without a transfection reagent (lipofectomine) and GalNAc-modified siHOTAIR was administered to the human retinal endothelial cell culture with and without a transfection agent. Cells treated with scrambled oligonucleotides (SCR) or conventional siRNA without transfection reagent showed significant glucose-induced upregulation of HOTAIR mRNA (FIG. 4) and VEGFA mRNA (FIG. 5). However, GalNAc siHOTAIR prevented high glucose-mediated HOTAIR mRNA (FIG. 4) induction and VEGFA mRNA (FIG. 5) without transfection reagent. When the cells were treated with transfection reagent, only cells transfected with SCR had a high glucose-mediated induction of HOTAIR and VEGFA, while both conventional and GalNAc-modified siHOTAIR were able to prevent the upregulation (see FIGS. 4 and 5).

siHOTAIR modified with GalNAc (GalNAc-siHOTAIR) is more efficiently taken up by the HRECs compared to unmodified siHOTAIR with transfection reagent, and do not to require transfection reagent.

The effectiveness of siHOTAIR in HREC was measured by a decrease in the level of its lncRNA HOTAIR target, in addition to the other pro-angiogenic molecules that also appear to be regulated by HOTAIR, such as VEGFA (VEGF) (see prior publications). GalNAc-siHOTAIR was more effective at suppressing VEGF mRNA levels compared to unmodified siHOTAIR (FIG. 5). Furthermore, GalNAc-modified siRNAs against regulatory lncRNAs (i.e. GalNAc siHOTAIR, GalNAc siMALAT1) is as effective at knocking down VEGFA as GalNAc-modified siRNAs designed specifically against VEGF (GalNAc siVEGF) (see FIG. 6).

5 nM, 10 nM, 25 nM, 50 nM and 100 nM levels of GalNAc-siHOTAIR were similarly effective at reducing HOTAIR lncRNA levels (FIG. 7) and VEGFA levels (FIG. 8) in cell culture.

As illustrated in FIG. 10, unmodified siHOTAIR (also referred to as naked siHOTAIR) is a robust and long-lasting blocker of VEGFA (VEGF) compared to Lucentis®, a prescription medicine for the treatment of patients with wet age-related macular degeneration (wAMD), diabetic retinopathy (DR), and diabetic macular edema (DME). As shown in FIG. 10, GalNAc siHOTAIR is even stronger than both Lucentis and unmodified siHOTAIR at reducing upregulation of VEGFA.

FIG. 11 shows that unmodified siHOTAIR is also a robust and long-lasting blocker of angiogenic molecule ANGPTL4 compared to Lucentis® (Lucentis® was unable to reduce ANGPTL4 compared to HG+SCR). GalNAc siHOTAIR is even stronger than unmodified siHOTAIR at reducing ANGPTL4.

siHOTAIR is also a robust blocker of VEGF-C. FIG. 12 shows significantly reduced levels of VEGFC mRNA in human retinal endothelial cells when exposed to unmodified (HOTAIRsi) and GalNAc modified siHOTAIR, while Lucentis was unable to reduce the levels of VEGFC mRNA. No differences were observed between these two interventions.

GalNAc siHOTAIR is effective at preventing high glucose induced upregulation of VEGFA mRNA (FIG. 10) and VEGFC mRNA (FIG. 12), as well as of VEGFA protein (FIG. 13A) and VEGFC protein (FIG. 13B).

GalNAc siHOTAIR prevented high glucose-mediated inhibition of miR-200b (FIG. 14A) and miR-146a (FIG. 14B) in human retinal endothelial cells.

GalNAc siHOTAIR prevented high glucose-induced increase in permeability in human retinal endothelial cells (FIG. 15) and high glucose induced angiogenesis (FIG. 16).

The impact of GalNAc-siHOTAIR in HREC culture could be observed at both Day 4 (FIG. 9A) and Day 7 (FIG. 9B) post-treatment without the use of a transfection agent. Therefore, the effect of GalNAc-siHOTAIR is sustainable over time.

### Overview of Animal Results

The unexpected and surprising findings in vitro that GalNAc modified siRNA efficiently transfects into human retinal endothelial cells in the absence of transfection reagent and that it was more effective at suppressing VEGF mRNA levels compared to unmodified siHOTAIR and similarly effective at reducing HOTAIR lncRNA when compared to siHOTAIR, lead to testing the effectiveness of the GalNAc modified siRNA of the present disclosure in vivo by both intravitreal injection and topical administration with eye drops in two different animal species: mouse and rat.
1. Basic effects: (a) retinal localization of GalNAc-modified siRNAs; (b) Lack of toxicity; (c) Inhibition of angiogenesis.
2. Treatment: In both mouse and rats (a) GalNAc-modified siRNA has effects in the retina when delivered without transfection reagent (as an eyedrop), while unmodified siRNA does not have an effect in the retina; (b) GalNAc-modified siRNA works nearly equally well when administered via either retinal injection or eye drop.
3. Prevention: In both mouse and rats GalNAc-modified siRNA prevents diabetes-induced effects.
   GalNAc Modification Helps Target siRNA Specifically to the Retina

As illustrated in FIGS. 17A-17F, administration of eye drops having the GalNAc-modified siHOTAIR help in targeting the siRNA specifically to the retina. The nuclear stain, DAPI shows abundant fluorescence in the cornea and lens (17A), ciliary body (17B), and retina (17C). FAM, tagged to GalNAc-modified siRNA, showed strong fluorescence only in the retina (17F), and not in the cornea and lens (17D), or ciliary body (17E), indicating specific localization of the GalNAc-modified siRNA to the retina.

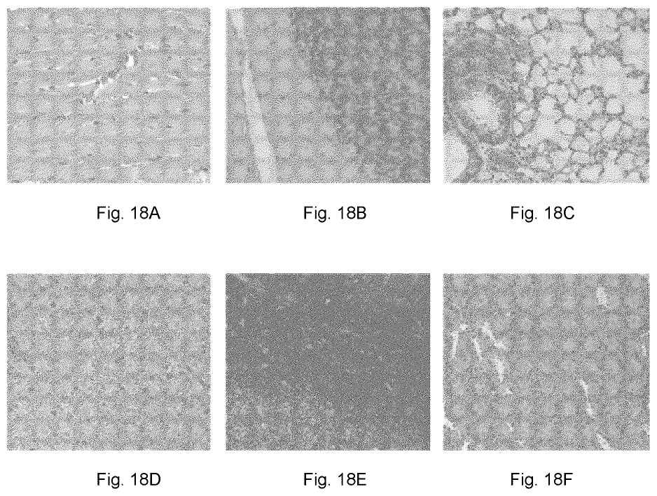

Importantly in vivo treatment with GalNAc siHOTAIR produced no histological evidence of toxicity in the heart (FIG. 18A), brain (18B), lung (18C), liver (18D), thymus (18E), or kidney (18F) following 5 consecutive days of eye drop treatments of diabetic mice. Table 3 illustrates the biological indicators of liver and renal toxicity that were measured.

TABLE 3
Biochemical indicators of liver and renal toxicity

| | Alanine | Aspartate | |
|---|---|---|---|
| | Transaminase | Transaminase | Creatinine |
| | (U/L) | (U/L) | (pmol/L) |
| Non-diabetic | 27-79 | 44-137 | 5-9 |
| Diabetic | 44-99 | 48-112 | 3-16 |
| Diabetic + injection | 49-87 | 56-189 | 4-7 |
| Diabetic + eye drop | 34-83 | 44-88 | 4-14 |
| Reference Ranges | 22-133 | 46-221 | 2.6-130 |

Effect of GalNAc siHOTAIR on Angiogenesis in Mice

As shown in FIGS. 19A-19B, GalNAc-siHOTAIR easily enters the retina of mice to inhibit production of VEGF. Matrigel plugs in mice with VEGFA(VEGF) showed recruitment of ECs, an early sign of angiogenesis (FIG. 19A), while plugs with VEGF and GalNAc-modified siHOTAIR without any transfection reagent showed much less recruitment of ECs, and no signs of angiogenesis (FIG. 19B). Although unmodified siRNA HOTAIR showed similar effects, it needed transfection with lipofectamine (not shown).

GalNAc siHOTAIR Treatment for Diabetic Retinopathy-Related Changes in Mice

STZ induced diabetic mice (D) were treated with a drop of (100 μM) GalNAc-modified HOTAIR siRNA in one eye twice daily for five days. Other eye of the D mice received diluent (distilled water). Retinal tissues were harvested and examined. No toxicity was noted.

As shown in FIGS. 20A and 20B, GalNAc-modified siHOTAIR eyedrop significantly reduced expression of retinal HOTAIR lncRNA expression (FIG. 20A) and mRNA expression of angiogenic molecules VEGFA (FIG. 20B), VEGFC (FIG. 21B) and ANGPTL4 (FIG. 21A) in the STZ induced diabetic (D) mice.

GalNAc siHOTAIR significantly reduced diabetes-induced HOTAIR lncRNA overexpression (FIG. 22A), VEGFA mRNA overexpression (FIG. 22B), VEGFC overexpression (FIG. 23A) and ANGPTL4 mRNA overexpression (FIG. 23B) both when delivered via intravitreal injection or when delivered as an eye drop.

GalNAc siHOTAIR Treatment for Diabetic Retinopathy-Related Changes in Rats

For injection treatment, diabetic rats were given 1 injection (10 μL) of GalNAc siHOTAIR and sacrificed one week following treatment. For eye drop treatment, diabetic rats were given 1 eye drop (5 μL) of GalNAc siHOTAIR per day for 1 week and then sacrificed. Retinal tissues were harvested and examined. No toxicity was noted.

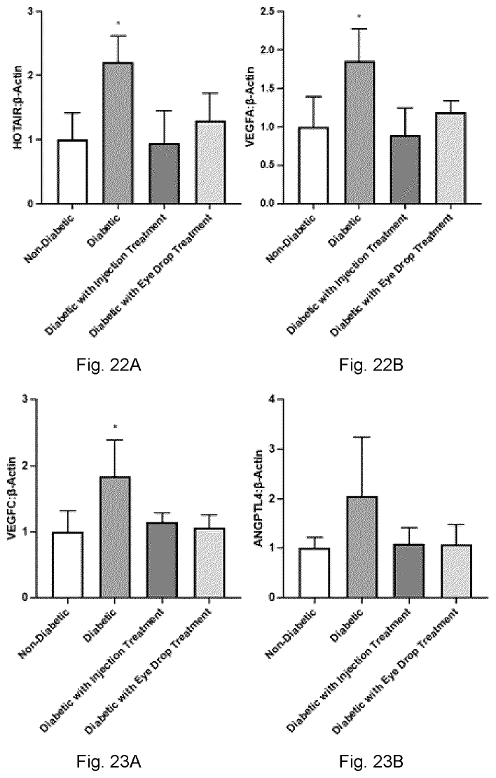

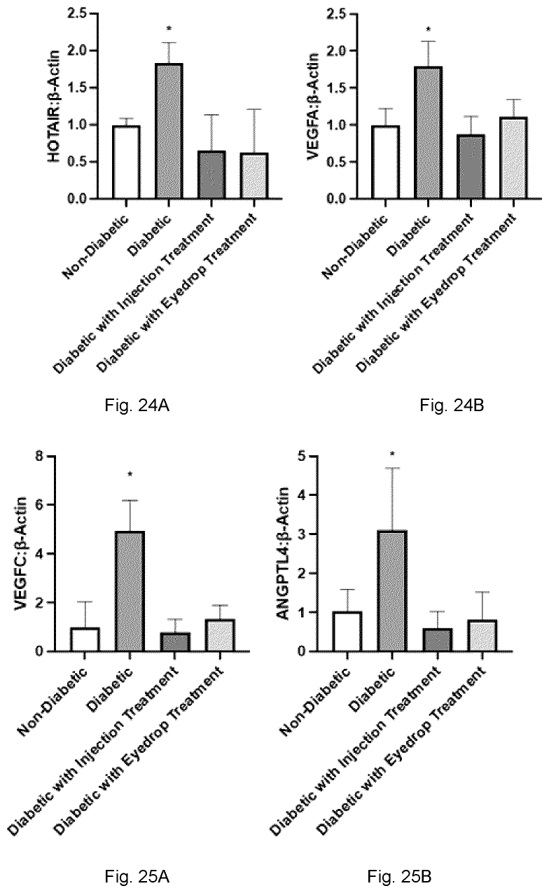

GalNAc siHOTAIR delivered either via injection or via eye drops reversed diabetes-induced upregulation of HOTAIR lncRNA (FIG. 24A), VEGFA mRNA (FIG. 24B), VEGFC mRNA (FIG. 25A) and ANGPTL4 mRNA (FIG. 25B) in the retinas of diabetic rats.

GalNAc siHOTAIR for Prevention of Diabetic Retinopathy-Related Changes in Mice

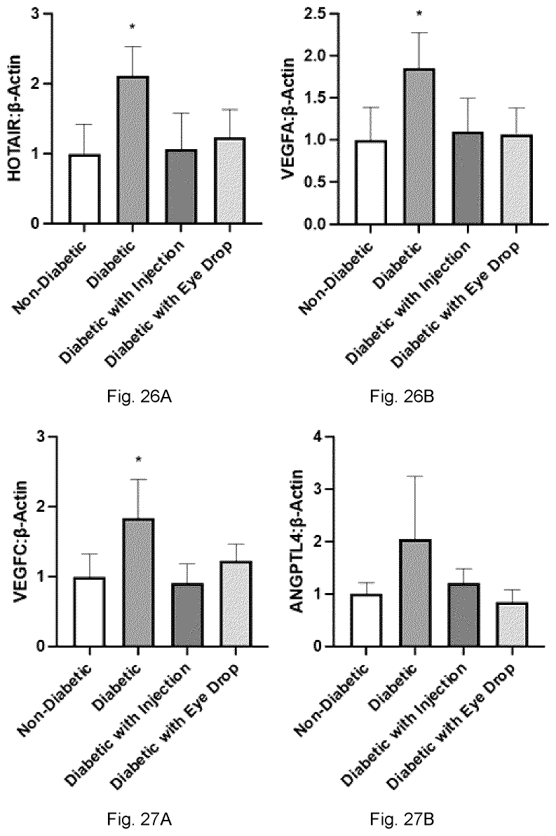

GalNAc siHOTAIR delivered via weekly injection or daily eye drops, prevented diabetes-induced upregulation of HOTAIR lncRNA (FIG. 26A), VEGFA mRNA (FIG. 26B), VEGFC mRNA (FIG. 27A) and ANGPTL4 (FIG. 27B) mRNA in the retinas of diabetic mice.

GalNAc siHOTAIR for Prevention of Diabetic Retinopathy-Related Changes in Rats

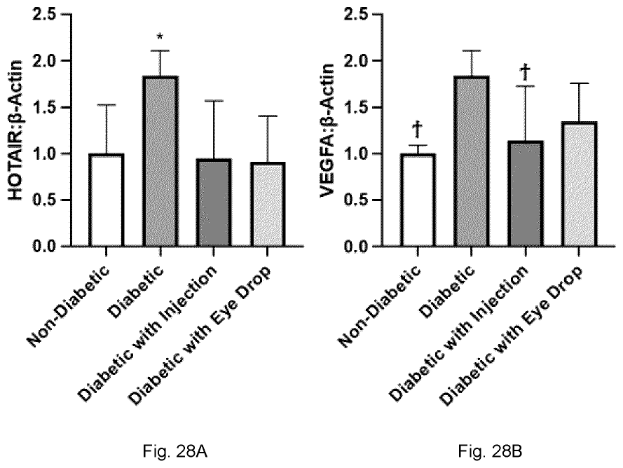

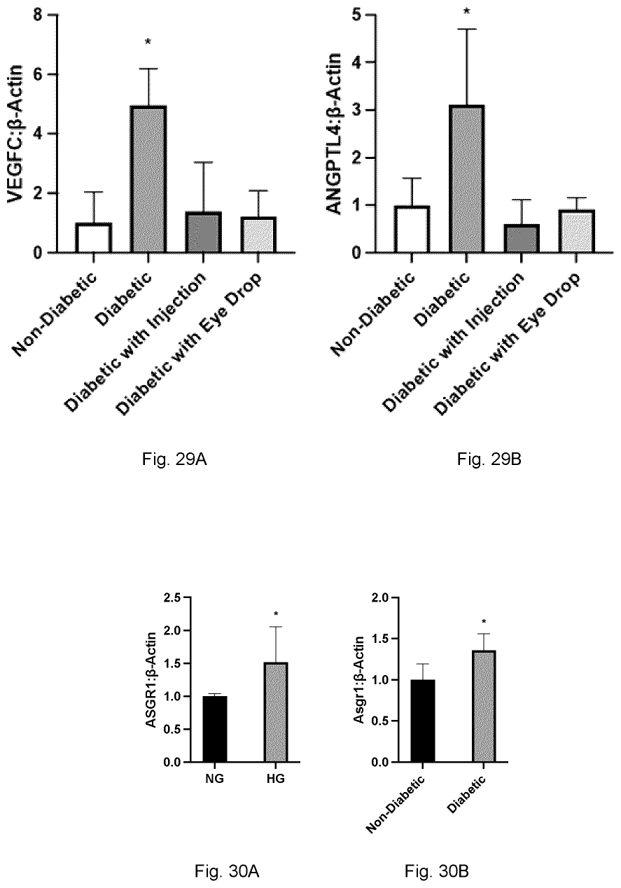

GalNAc siHOTAIR, delivered either via weekly injection or daily eye drops, prevented diabetes-induced upregulation of HOTAIR lncRNA (FIG. 28A), VEGFA mRNA (FIG. 28B), VEGFC mRNA (FIG. 29A) and ANGPTL4 (FIG. 29B) mRNA in the retinas of diabetic rats.

Table 4 lists the advantages of GalNAc-modified siHOTAIR over unmodified HOTAIR siRNA.

TABLE 4

| | Unmodified HOTAIR | |
|---|---|---|
| Effect | siRNA | GalNAc modified |
| Need for transfection | Yes | No |
| reagent | | |
| Efficacy | Less robust | More robust |
| Effect of Intravitreal | Good | Better |
| injection | | |
| Duration of HOTAIR | Less | More |
| inhibitory effect | | |
| Efficacy as an eyedrop | No | Yes, with significant |
| | | effects |
| Matrigel angiogenesis | Yes, with lipofectamine | Yes, Lipofectamine is not |
| assay | (not shown) | needed |
| Toxicity | None observed | None observed |

GalNAc modified HOTAIR siRNA can be used without transfection reagent both in vivo and in vitro, is more robust in preventing glucose/diabetes induced increased HOTAIR, VEGFA, ANGPTL4 and VEGFC overexpression both in vivo and in vitro, is devoid of toxicity, and can be used as a topical application.

TABLE 5
Human (see WO 2022/006667)
Detailed information for the 4 Human custom siHOTAIR sequences designed
by us. The oligonucleotides were converted to a 2′-hydroxyl, annealed, and
desalted duplex. SB = Internal custom design identification number.

| Custom siRNAs for Human HOTAIR: | Sequences and SEQ ID: |
|---|---|
| SiHOTAIR SB1 | Sense Strand: |
| Length: 21 nucleotides | 5′-CCAAAGAGUCUGAUGUUUACA-3′ |
| Mol. Wt. 13, 378.1 (g/mol) | (SEQ ID: 21) |
| Ext. Coeff 381, 186 (L/mol. cm) | Antisense Strand: |
| Target Sequence: | 5′-UAAACAUCAGACUCUUUGGGG-3′ |
| CCCCAAAGAGTCTGATGTTTACA (SEQ | (SEQ ID: 22) |
| ID NO: 29) | |
| (target position: 457-479) | |
| SiHOTAIR SB2 | Sense Strand: |
| Length: 21 nucleotides | 5′-CAUAAACAAUAUAUCUGUUGG-3′ |
| Mol. Wt. 13, 373.1 (g/mol) | (SEQ ID: 23) |
| Ext. Coeff 402, 101 (L/mol. cm) | Antisense Strand: |
| Target Sequence: | 5′-AACAGAUAUAUUGUUUAUGAG-3′ |
| CTCATAAACAATATATCTGTTGG | (SEQ ID: 24) |
| (SEQ ID NO: 30) | |
| (target position: 1156-1178) | |
| siHOTAIR SB3 | Sense Strand: |
| Length: 21 nucleotides | 5′-CUCUAUAAUAUGCUUAUAUUA-3′ |
| Mol. Wt. 13, 256.0 (g/mol) | (SEQ ID: 25) |
| Ext. Coeff 405, 216 (L/mol.cm) | Antisense Strand: |
| Target Sequence: | 5′-AUAUAAGCAUAUUAUAGAGUU-3′ |
| AACTCTATAATATGCTTATATTA (SEQ | (SEQ ID: 26) |
| ID NO: 31) | |
| (target position: 1700-1722) | |
| siHOTAIR SB4 | Sense Strand: |
| Length: 21 nucleotides | 5′-GUGUAUAUAUAAUAAUGUAUU-3′ |
| Mol. Wt. 13, 264.0 (g/mol) | (SEQ ID: 27) |
| Ext. Coeff 413, 849 (L/mol.cm) | Antisense Strand: |
| Target Sequence: | 5′-UACAUUAUUAUAUAUACACAA-3′ |
| TTGTGTATATATAATAATGTATT (SEQ | (SEQ ID: 28) |
| ID NO: 32) | |
| (target position: 2160-2182) | |

Furthermore, custom double-stranded siRNAs targeting specific regions of HOTAIR (near 5′ end, middle gene body, and near 3′ end) were developed and HRECs were subsequently transfected. Compared to SCR HG controls, HOTAIR expressions were reduced by −67%, −41%, −57%, and −32% using siHOTAIR SB1, siHOTAIR SB2, siHOTAIR SB3, and siHOTAIR SB4, respectively. The knockdown of HOTAIR also directly influenced the transcript levels of VEGF-A, ET-1 and ANGPTL4, albeit variable reductions existing for each siRNA (greatest reductions were observed for the siRNAs designed to target near the 5′ end of HOTAIR: SB1 and SB2). SB1 was used for subsequent experiments and GalNAc modification was done on SB1.

TABLE 6
Mouse HOTAIR siRNAs
Detailed information for the 4 Mouse custom siHOTAIR sequences designed
by us. The oligonucleotides were converted to a 2′-hydroxyl, annealed,
duplex. BF = Internal custom design identification number.

| Custom siRNAs for mouse HOTAIR: | Sequences and SEQ ID: |
|---|---|
| Target position: 31-53 BF1 | Antisense/Guide = |
| Target sequence+ 2 nt overhang: | UAAAGAAAACUUCAUUUACAG |
| CTGTAAATGAAGTTTTCTTTATG | (SEQ ID: 33) |
| Tm guide: 5.5° C. | Sense/Passenger = |
| Tm passenger: 8.9° C. | GUAAAUGAAGUUUUCUUUAUG |
| (SEQ ID: 41) | (SEQ ID: 34) |
| Target position: 1075-1097 BF2 | Antisense/Guide = |
| Target sequence+ 2 nt overhang: | UAUAGAAAAAUUCUUUAAGAG |
| CTCTTAAAGAATTTTTCTATAGT | (SEQ ID: 35) |
| Tm guide: 6.9° C. | Sense/Passenger = |
| Tm passenger: 7.1° C. | CUUAAAGAAUUUUUCUAUAGU |
| (SEQ ID: 42) | (SEQ ID: 36) |
| Target position: 1908-1930 BF3 | Antisense/Guide = |
| Target sequence+ 2 nt overhang: | UUUUAAAAAUAAAUAUUGGAG |
| CTCCAATATTTATTTTTAAAAAA | (SEQ ID: 37) |
| Tm guide: −9.1° C. | Sense/Passenger = |
| Tm passenger: −1.8° C. | CCAAUAUUUAUUUUUAAAAAA |
| (SEQ ID: 43) | (SEQ ID: 38) |
| Target position: 2035-2057 BF4 | Antisense/Guide = |
| Target sequence+ 2 nt overhang: | AAUUAUAUCACAUUCUUACAC |
| GTGTAAGAATGTGATATAATTCT | (SEQ ID: 39) |
| Tm guide: −8.6° C. | Sense/Passenger = |
| Tm passenger: 6.9° C. | GUAAGAAUGUGAUAUAAUUCU |
| (SEQ ID: 44) | (SEQ ID: 40) |

Furthermore, custom double-stranded siRNAs targeting specific regions of HOTAIR (near 5′ end, middle gene body, and near 3′ end) were developed and tested on mouse endothelial cells. Compared to SCR HG controls, highest HOTAIR expression reduction was obtained using BF3. The knockdown of HOTAIR also directly influenced the transcript levels of VEGF-A and ANGPTL4, albeit variable reductions existing for each siRNA.

BF3 was used for subsequent experiments and GalNAc modification was done on BF3.

Other Sequences Used in this Disclosure

Human siVEGFA:
Sense
(SEQ ID NO: 45)
GGAGUACCCUGAUGAGAUCTT
Antisense
(SEQ ID NO: 46)
GAUCUCAUCAGGGUACUCCTT
Human siMALAT1:
Sense/Passenger:
(SEQ ID NO: 47)
CUGUUAAGAAAAAUCUAGAAA
Antisense/Guide:
(SEQ ID NO: 48)
UCUAGAUUUUUCUUAACAGCU
MOUSE ASGR1-F2:
(SEQ ID NO: 49)
5′-TTCCAGCACCTGGACAATGA-3′
MOUSE ASGR1-R2:
(SEQ ID NO: 50)
5′-ACAGACAACCACCAGCAACA-3′
HUMAN ASGR1-F:
(SEQ ID NO: 51)
5′-GAGAGTGACCACCATCAGCT-3′
HUMAN ASGR1-R:
(SEQ ID NO: 52)
5′-TCGCTGTGAAGTTGCTGAAC-3′

### Example 2—GalNAc-siANRIL, GalNAc-siMALAT1, GalNAc-siWISPER and GalNAc-siZFAS1

HREC cultures were treated with the following treatment groups: normal glucose (5 mM glucose, NG) plus scrambled (Scr) siRNA, high glucose (25 mM, HG) plus Scr (HG+Scr), HG plus each of siANRIL, siMALAT1, siWISPER, siH19, siMIAT and siZFAS1 with lipofectomine as transfection reagent, HG plus each of siANRIL, siMALAT1, siWISPER, siH19, siMIAT and siZFAS1 without transfection reagent, HG plus GalNAc-siANRIL, GalNAc-siMALAT1, GalNAc-siWISPER, GalNAc-siH19, GalNAc-siMIAT and GalNAc-siZFAS1 with transfection reagent, and HG plus GalNAc-siANRIL, GalNAc-siMALAT1, GalNAc-siWISPER, GalNAc-siH19, GalNAc-siMIAT and GalNAc-siZFAS1 without transfection reagent. Two dosages of each siRNA (modified and unmodified) were used: 50 nm and 100 nm.

For eye drop studies, STZ induced diabetic mice (obtained as described above in Example 1) were treated with a drop of GalNAc-modified siANRIL, GalNAc-siMALAT1, GalNAc-siWISPER, GalNAc-siH19, GalNAc-siMIAT and GalNAc-siZFAS1 in one eye twice daily for five days. Other eye of the diabetic mice received diluent (distilled water). Retinal tissues were harvested and examined. No toxicity was noted.

Levels of ANRIL lncRNA, MALAT1 lncRNA, WISPER lncRNA, H19 lncRNA, MIAT lncRNA, ZFAS1 lncRNA, VEGFA (VEGF) mRNA, VEGF C mRNA and ANGPTL4 mRNA were measured for each of the treatment groups to demonstrate superiority of the GalNAc modified siRNAs at reducing levels of the lncRNA, VEGFA (VEGF) mRNA, VEGF C mRNA and ANGPTL4 mRNA in HREC cultures and in the harvested retinal tissues over their respective unmodified versions.

TABLE 7

| Target | Sequence |
|---|---|
| hAnril siRNA-1 | Antisense/Guide: |
| Target position: 983-1005 | AAAUGUUUCUAAUAAUUCCAU |
| target sequence 21 nt target + 2 nt overhang: | (SEQ ID NO: 53) |
| ATGGAATTATTAGAAACATTTTC | Sense/Passenger: |
| (SEQ ID NO: 64) | GGAAUUAUUAGAAACAUUUUC |
| seed-duplex stability (Tm): guide 6.9° C.; | (SEQ ID NO: 54) |
| passenger 7.4° C. | |
| hAnril siRNA-2 | Antisense/Guide: |
| Target position: 1468-1490 | UUAUUCUUCUCAUCAAAAGGG |
| target sequence 21 nt target + 2 nt overhang: | (SEQ ID NO: 55) |
| CCCTTTTGATGAGAAGAATAAGC | Sense/Passenger: |
| (SEQ ID NO: 65) | CUUUUGAUGAGAAGAAUAAGC |
| seed-duplex stability (Tm): guide 5.3° C.; | (SEQ ID NO: 56) |
| passenger 1.8° C. | |
| hAnril siRNA-3 | Antisense/Guide: |
| Target position: 3151-3173 | UCUGAUUUGCAAAAACAGCUG |
| target sequence 21 nt target + 2 nt overhang: | (SEQ ID NO: 57) |
| CAGCTGTTTTTGCAAATCAGATT | Sense/Passenger: |
| (SEQ ID NO: 66) | GCUGUUUUUGCAAAUCAGAUU |
| seed-duplex stability (Tm): guide 12.0° C.; | (SEQ ID NO: 58) |
| passenger 10.3° C. | |
| hMalat1 siRNA-1 | Antisense/Guide: |
| Target position: 475-497 | UAAAGUAGACCAACUAAGCGA |
| target sequence 21 nt target + 2 nt overhang: | (SEQ ID NO: 59) |
| TCGCTTAGTTGGTCTACTTTAAA | Sense/Passenger: |
| (SEQ ID NO: 67) | GCUUAGUUGGUCUACUUUAAA |
| seed-duplex stability (Tm): guide 9.8° C.; | (SEQ ID NO: 60) |
| passenger 9.8° C. | |
| hMalat1 siRNA-2 | Antisense/Guide: |
| Target position: 1153-1175 | UCUAGAUUUUUCUUAACAGCU |
| target sequence 21 nt target + 2nt overhang: | Sense/Passenger: |
| AGCTGTTAAGAAAAATCTAGAAA | CUGUUAAGAAAAAUCUAGAAA |
| (SEQ ID NO: 68) | (SEQ ID NO: 61) |
| seed-duplex stability (Tm): guide 11.6° C.; | |
| passenger 11.8° C. | |
| hMalat1 siRNA-3 | Antisense/Guide: |
| Target position: 1400-1422 | UUAAGUAGUUGUAUUAAUCU |
| target sequence 21 nt target + 2 nt overhang: | (SEQ ID NO: 62) |
| GAGATTAATACAACTACTTAAAA | Sense/Passenger: |
| (SEQ ID NO: 69) | GAUUAAUACAACUACUUAAAA |
| seed-duplex stability (Tm): guide 11.3º C.; | (SEQ ID NO: 63) |
| passenger −8.0º C. | |
| hHULC | No sequence available |
| Commercially obtained (ThermoFisher | |
| Scientific n272671) | |
| hZFAS1 | No sequence available |
| Commercially obtained (ThermoFisher | |
| Scientific n272231) | |

### REFERENCES

### References of Background and Description of the Drawings

1. International Diabetes Federation. IDF Diabtes Atlas. (Brussels, Belgium, 2021).
2. Saeedi, P. et al. Global and regional diabetes prevalence estimates for 2019 and projections for 2030 and 2045: Results from the International Diabetes Federation Diabetes Atlas, 9th edition. Diabetes Res Clin Pract 157, 107843 (2019).
3. Leasher, J. L. et al. Global Estimates on the Number of People Blind or Visually Impaired by Diabetic Retinopathy: A Meta-analysis From 1990 to 2010. Dia Care 39, 1643-1649 (2016).
4. Chua, J., Lim, C. X. Y., Wong, T. Y. & Sabanayagam, C. Diabetic retinopathy in the Asia-pacific. Asia-Pacific Journal of Ophthalmology vol. 7 3-16 (2018).
5. Teo, Z. L. et al. Global Prevalence of Diabetic Retinopathy and Projection of Burden through 2045. Ophthalmology S0161642021003213 (2021) doi:10.1016/j.ophtha.2021.04.027.
6. Ding, J. & Wong, T. Y. Current Epidemiology of Diabetic Retinopathy and Diabetic Macular Edema. Curr Diab Rep 12, 346-354 (2012).
7. Wang, W. & Lo, A. Diabetic Retinopathy: Pathophysiology and Treatments. IJMS 19, 1816 (2018).
8. Gui, F., You, Z., Fu, S., Wu, H. & Zhang, Y. Endothelial Dysfunction in Diabetic Retinopathy. Front Endocrinol (Lausanne) 11, 591 (2020).
9. Cheung, N., Mitchell, P. & Wong, T. Y. Diabetic retinopathy. Lancet 376, 124-136 (2010).
10. Tarr, J. M., Kaul, K., Chopra, M., Kohner, E. M. & Chibber, R. Pathophysiology of Diabetic Retinopathy. ISRN Ophthalmology 2013, 1-13 (2013).
11. Lechner, J., O'Leary, 0. E. & Stitt, A. W. The pathology associated with diabetic retinopathy. Vision Research 139, 7-14 (2017).
12. Duh, E. J., Sun, J. K. & Stitt, A. W. Diabetic retinopathy: current understanding, mechanisms, and treatment strategies. JCI Insight 2, (2017).
13. Ferrara, N., Gerber, H.-P. & LeCouter, J. The biology of VEGF and its receptors. Nat Med 9, 669-676 (2003).
14. Gonzalez-Cortes, J. H. et al. Current Treatments and Innovations in Diabetic Retinopathy and Diabetic Macular Edema. Pharmaceutics 15, 122 (2023).
15. van Wijngaarden, P., Coster, D. J. & Williams, K. A. Inhibitors of Ocular NeovascularizationPromises and Potential Problems. JAMA 293, 1509-1513 (2005).
16. Kornienko, A. E., Guenzl, P. M., Barlow, D. P. & Pauler, F. M. Gene regulation by the act of long non-coding RNA transcription. BMC Biology 11, 59 (2013).
17. Yoon, J.-H., Abdelmohsen, K. & Gorospe, M. Functional interactions among microRNAs and long noncoding RNAs. Seminars in Cell & Developmental Biology 34, 9-14 (2014).
18. Wu, Z. et al. Regulation of lncRNA expression. Cell Mol Biol Lett 19, 561-575 (2014).
19. Gong, Q. & Su, G. Roles of miRNAs and long noncoding RNAs in the progression of diabetic retinopathy. Bioscience Reports 37, BSR20171157 (2017).
20. Yoon, J.-H. et al. Scaffold function of long non-coding RNA HOTAIR in protein ubiquitination. Nat Commun 4, 2939 (2013).
21. Biswas, S. et al. MALAT1: An Epigenetic Regulator of Inflammation in Diabetic Retinopathy. Sci Rep 8, 6526 (2018).
22. Thomas, A. A., Feng, B. & Chakrabarti, S. ANRIL: A Regulator of VEGF in Diabetic Retinopathy. Invest. Ophthalmol. Vis. Sci. 58, 470 (2017).
23. Kanasty, R., et al., Delivery materials for siRNA therapeutics. Nature Materials, 12, 967-977 (2013).
24. WO2016055601.
25. WO2022076922.
26. Bouckenheimer, J., Assou, S., Riquier, S., Hou, C., Philippe, N., Sansac, C., De Vos, J. (2016). Long non-coding RNAs in human early embryonic development and their potential in ART. Human Reproduction Update, 23(1), 19-40. http://doi.org/10.1093/humupd/dmw035
27. Prensner, J. R., & Chinnaiyan, A. M. (2011). The emergence of lncRNAs in cancer biology. Cancer Discovery. http://doi.org/10.1158/2159-8290.CD-11-0209
28. Wang, D. Q., Fu, P., Yao, C., Zhu, L. S., Hou, T. Y., Chen, J. G., . . . Zhu, L. Q. (2018). Long Non-coding RNAs, Novel Culprits, or Bodyguards in Neurodegenerative Diseases. Molecular Therapy—Nucleic Acids, 10, 269-276.
29. Hirata, H.; Hinoda, Y.; Shahryari, V.; Deng, G.; Nakajima, K.; Tabatabai, Z. L.; Ishii, N.; Dahiya, R. Long noncoding RNA MALAT1 promotes aggressive renal cell carcinoma through Ezh2 and interacts with miR-205. Cancer Res. 2015, 75, 1322-1331
30. Ren, S.; Liu, Y.; Xu, W.; Sun, Y.; Lu, J.; Wang, F.; Wei, M.; Shen, J.; Hou, J.; Gao, X.; et al. Long noncoding RNA MALAT-1 is a new potential therapeutic target for castration resistant prostate cancer. J. Urol. 2013,190, 2278-2287.
31. Zhang, Q. S.; Wang, Z. H.; Zhang, J. L.; Duan, Y. L.; Li, G. F.; Zheng, D. L. Beta-asarone protects against MPTP-induced Parkinson's disease via regulating long non-coding RNA MALAT1 and inhibiting a-synuclein protein expression. Biomed.

### Pharmacother. 2016, 83, 153-159.

32. Vausort, M.; Wagner, D. R.; Devaux, Y. Long noncoding RNAs in patients with acute myocardial infarction. Circ. Res. 2014, 115, 668-677.
33. Pachnis, V.; Belayew, A.; Tilghman, S. M. Locus unlinked to alpha-fetoprotein under the control of the murine raf and Rif genes. Proc. Natl. Acad. Sci. USA 2006, 81, 5523-5527.
34. Micheletti, R. et al., 2017. The long noncoding RNA Wisper controls cardiac fibrosis and remodeling. Science Translational Medicine, 9(395).
35. Askarian-Amiri M E, Crawford J, Franch J D, Smart C E, Smith M A, Clark M B, Ru K, M T M, Thompson E R, Lakhani S R, Vargas A C, Campbell I G, Brown M A, Dinger M E, Mattick J S. SNORD-host RNA Zfasl is a regulator of mammary development and a potential marker for breast cancer. RNA., 2011. 17:878-891.
36. Faltejskova-Vychytilova P, Hombach S, Mlcochova J, Kretz M, Svoboda M, Slaby O. Long non-coding RNA ZFAS1 interacts with CDK1 and is involved in p53-dependent cell cycle control and apoptosis in colorectal cancer. Oncotarget., 2016. 7:622-637.
37. Panzitt K, Tschernatsch M M, Guelly C, Moustafa T, Stradner M, Strohmaier H M, Buck C R, Denk H, Schroeder R, Trauner M, Zatloukal K. Characterization of HULC, a novel gene with striking up-regulation in hepatocellular carcinoma, as noncoding RNA. Gastroentology., 2007. 132:330-342.
38. Ishii, N.; Ozaki, K.; Sato, H.; Mizuno, H.; Saito, S.; Takahashi, A.; Miyamoto, Y.; Ikegawa, S.; Kamatani, N.; Hori, M.; et al. Identification of a novel non-coding RNA, MIAT, that confers risk of myocardial infarction. J. Hum. Genet. 2006, 51, 1087-1099.
39. Barry, G.; Briggs, J. A.; Vanichkina, D. P.; Poth, E. M.; Beveridge, N. J.; Ratnu, V. S.; Nayler, S. P.; Nones, K.; Hu, J.; Bredy, T. W.; et al. The long non-coding RNA Gomafu is acutely regulated in response to neuronal activation and involved in schizophrenia-associated alternative splicing. Mol. Psychiatry 2014, 19, 486-494.
40. Zhang, H. Y.; Zheng, F. S.; Yang, W.; Lu, J. Bin. The long non-coding RNA MIAT regulates zinc finger E-box binding homeobox 1 expression by sponging miR-150 and promoting cell invasion in non-small-cell lung cancer. Gene 2017, 633, 61-65.
41. Blackshaw, S.; Harpavat, S.; Trimarchi, J.; Cai, L.; Huang, H.; Kuo, W. P.; Weber, G.; Lee, K.; Fraioli, R. E.; Cho, S. H.; et al. Genomic analysis of mouse retinal development. PLoS Biol. 2004, 2, 1-21.
42. Aprea, J.; Prenninger, S.; Dori, M.; Ghosh, T.; Monasor, L. S.; Wessendorf, E.; Zocher, S.; Massalini, S.; Alexopoulou, D.; Lesche, M.; et al. Transcriptome sequencing during mouse brain development identifies long non-coding RNAs functionally involved in neurogenic commitment. EMBO J. 2013, 32, 3145-3160.
43. Shen, Y.; Dong, L. F.; Zhou, R. M.; Yao, J.; Song, Y. C.; Yang, H.; Jiang, Q.; Yan, B. Role of long non-coding RNA MIAT in proliferation, apoptosis and migration of lens epithelial cells: A clinical and in vitro study. J. Cell Mol. Med. 2016, 20, 537-548.
44. Pasmant, E.; Laurendeau, I.; Heron, D.; Vidaud, M.; Vidaud, D.; Bieche, I. Characterization of a germ-line deletion, including the entire INK4/ARF locus, in a melanoma-neural system tumor family: Identification of ANRIL, an antisense noncoding RNA whose expression coclusters with ARF. Cancer Res. 2007, 67, 3963-3969.
45. Broadbent, H. M.; Peden, J. F.; Lorkowski, S.; Goel, A.; Ongen, H.; Green, F.; Clarke, R.; Collins, R.; Franzosi, M. G.; Tognoni, G.; et al. Susceptibility to coronary artery disease and diabetes is encoded by distinct, tightly linked SNPs in the ANRIL locus on chromosome 9p. Hum. Mol. Genet. 2008, 17, 806-814.
46. Li, Z.; Yu, X.; Shen, J. ANRIL: A pivotal tumor suppressor long non-coding RNA in human cancers. Tumor Biol. 2016, 37, 5657-5661.
47. Rein, D. B. et al. Prevalence of Age-Related Macular Degeneration in the US in 2019. JAMA Ophthalmology 140, 1202-1208 (2022).
48. Song, D., Liu, P., Shang, K. & Ma, Y. Application and mechanism of anti-VEGF drugs in age-related macular degeneration. Front Bioeng Biotechnol 10, 943915 (2022).
49. Zhang, R. et al. Roles and mechanisms of long non-coding RNAs in age-related macular degeneration. Heliyon 9, e22307 (2023).
50. Chen X, Rong S S, Xu Q, Tang F Y, Liu Y, Gu H, Tam P O, Chen L J, Brelen M E, Pang C P, Zhao C. Diabetes mellitus and risk of age-related macular degeneration: a systematic review and meta-analysis. PLoS One. 2014 Sep. 19; 9(9):e108196. doi: 10.1371/journal.pone.0108196. PMID: 25238063; PMCID: PMC4169602.

### REFERENCES FOR MATERIALS AND METHODS

1. Biswas S, Feng B, Chen S, Liu J, Aref-Eshghi E, Gonder J, Ngo V, Sadikovic B, Chakrabarti S. The Long Non-Coding RNA HOTAIR Is a Critical Epigenetic Mediator of Angiogenesis in Diabetic Retinopathy. Inv. Ophthalmol. Vis. Sc. 2021, Vol. 62, 20. doi: https://doi.org/10.1167/iovs.62.3.2055.
2. Biswas, S., Feng, B., Thomas, A., Chen, S., Aref-Eshghi, E., Sadikovic, B., & Chakrabarti, S. (2018). Endothelin-1 regulation is entangled in a complex web of epigenetic mechanisms in diabetes. Physiological Research, 67, S115-S125. http://doi.org/10.33549/physiolres.933836
3. Ruiz, M. A., Feng, B., & Chakrabarti, S. (2015). Polycomb repressive complex 2 regulates MiR-200b in retinal endothelial cells: Potential relevance in diabetic retinopathy. PLoS ONE, 10(4). http://doi.org/10.1371/journal.pone.0123987
4. Thomas, A. A., Biswas, S., Feng, B., Chen, S., Gonder, J., & Chakrabarti, S. (2019). lncRNA H19 prevents endothelial-mesenchymal transition in diabetic retinopathy. Diabetologia, 62(3), 517-530. http://doi.org/10.1007/s00125-018-4797-6
5. Biswas, S., Thomas, A. A., Chen, S., Aref-Eshghi, E., Feng, B., Gonder, J., . . . Chakrabarti, S. (2018). MALAT1: An Epigenetic Regulator of Inflammation in Diabetic Retinopathy. Scientific Reports, 8(1). http://doi.org/10.1038/s41598-018-24907-w
6. Liu, J., Chen, S., Biswas, S., Nagrani, N., Chu, Y., Chakrabarti, S., & Feng, B. (2020). Glucose-induced oxidative stress and accelerated aging in endothelial cells are mediated by the depletion of mitochondrial SIRTs. Physiological Reports, 8(3). http://doi.org/10.14814/phy2.14331
7. Thomas, A. A., Feng, B., & Chakrabarti, S. (2017). ANRIL: A regulator of VEGF in diabetic retinopathy. Investigative Ophthalmology and Visual Science, 58(1), 470-480. http://doi.org/10.1167/iovs.16-20569.
Vis Sci. 2019 Oct. 1; 60(13):4084-4096. doi: 10.1167/iovs.19-26767.
8. Turchinovich, A., Zoidl, G., & Dermietzel, R. (2010). Non-viral siRNA delivery into the mouse retina in vivo. BMC Ophthalmology, 10(1). http://doi.org/10.1186/1471-2415-10-25
9. Goytain A, Ng T. NanoString nCounter Technology: High-Throughput RNA Validation.
Methods Mol Biol. 2020; 2079:125-139. doi: 10.1007/978-1-4939-9904-0_10.
10. Aref Z, Quax P H. In Vivo Matrigel Plug Assay as a Potent Method to Investigate Specific Individual Contribution of Angiogenesis to Blood Flow Recovery in Mice. Int J Mol Sci. 2021: 8909.18. doi: 10.3390/ijms22168909
11. Wang X, Huang G, Mu J, Cong Z, Chen S, Fu D, Qi J, Li Z. Arrb2 promotes endothelial progenitor cell-mediated postischemic neovascularization. Theranostics 2020; 10(21):9899-9912. doi:10.7150/thno.45133. eCollection 2020.

Although various embodiments of the disclosure have been described and illustrated, it will be apparent to those skilled in the art in light of the present description that numerous modifications and variations can be made. The scope of the invention is defined more particularly in the appended claims. All publications, patents and patent applications referred to in this document are incorporated herein by reference. Where a term in the present application is found to be defined differently in a document incorporated herein by reference, the definition provided herein is to serve as the definition for the term.

1.-84. (canceled)

85. A method of treating or preventing an ocular disease in a subject comprising administering locally to an eye of the subject a nucleic acid (NA) molecule having an acetylated amino sugar moiety (CHO) (CHO-NA), wherein the NA is an inhibitor of a long-noncoding RNA (lncRNA) associated with the ocular disease, wherein the lncRNA is HOTAIR lncRNA.

86. The method of claim 85, wherein the nucleic acid molecule is a small interfering RNA (siRNA), a microRNA, a PlWI-interacting RNA (piRNA), a small nuclear RNA, an antisense nucleic acid, short hair-pin RNA (shRNA), or a ribozyme.

87. The method of claim 85, wherein the nucleic acid molecule is a siRNA.

88. The method of claim 85, wherein the CHO-NA is a HOTAIR siRNA having an N-acetylgalactosamine (GalNAc-siHOTAIR), and wherein the HOTAIR siRNA (siHOTAIR) comprises a sequence selected from SEQ ID NO: 22, SEQ ID NO: 24, SEQ ID NO: 26, and SEQ ID NO:28, or wherein the siHOTAIR comprises a sequence selected from SEQ ID NO: 21, SEQ ID NO: 23, SEQ ID NO: 25, and SEQ ID NO:27.

89. The method of claim 85, wherein the CHO is an N-acetylgalactosamine (GalNAc).

90. The method of claim 85, wherein the local administration is one of topical administration, subretinal injection, subconjunctival injection, intravitreal injection, or a combination thereof.

91. The method of claim 85, wherein the local administration comprises eye drops and hydrogels.

92. The method of claim 85, wherein the local administration of the nucleic acid molecule is free of a transfection agent.

93. The method of claim 85, wherein the CHO-NA is administered in combination with (i) a different CHO-NA, (ii) another agent known to treat or prevent the ocular disease, or (iii) both (i) and (ii).

94. The method of claim 85, wherein the ocular disease is an eye-related diabetes-induced condition.

95. The method of claim 85, wherein ocular disease is a retinal disease.

96. The method of claim 85, wherein the ocular disease includes dry age-related macular degeneration (AMD), wet AMD, non-proliferative diabetic retinopathy, proliferative diabetic retinopathy, neovascular glaucoma and/or ischemic retinopathy.

97. The method of claim 85, wherein the CHO is a ligand of a retinal asialoglycoprotein receptor (ASGR).

98. A nucleic acid molecule (NA) having an acetylated amino sugar moiety (CHO) (CHO-NA) for local use in treating or preventing an ocular disease, wherein the NA is an inhibitor of a long-noncoding RNA (lncRNA) associated with the ocular disease, wherein the lncRNA is HOTAIR lncRNA.

99. The CHO-NA for local use of claim 98, wherein the nucleic acid molecule is a small interfering RNA (siRNA), a microRNA, a PlWI-interacting RNA (piRNA), a small nuclear RNA (snRNA), a short hair-pin RNA (shRNA), an antisense nucleic acid, or a ribozyme.

100. The CHO-NA for local use of claim 98, wherein the nucleic acid molecule is a siRNA.

101. The CHO-NA for local use of claim 98, wherein the CHO-NA is a HOTAIR siRNA having an N-acetylgalactosamine (GalNAc-siHOTAIR), and wherein the HOTAIR siRNA (siHOTAIR) comprises a sequence selected from SEQ ID NO: 22, SEQ ID NO: 24, SEQ ID NO: 26, and SEQ ID NO:28, or wherein the siHOTAIR comprises a sequence selected from SEQ ID NO: 21, SEQ ID NO: 23, SEQ ID NO: 25, and SEQ ID NO:27.

102. The CHO-NA for local use of claim 98, wherein the CHO is an N-acetylgalactosamine (GalNAc).

103. The CHO-NA for local use of claim 98, wherein the ocular disease is dry age-related macular degeneration (AMD), wet AMD, nonproliferative diabetic retinopathy, proliferative diabetic retinopathy, neovascular glaucoma and/or ischemic retinopathy.

104. The CHO-NA for local use of claim 98, wherein the CHO is a ligand of a retinal asialoglycoprotein receptor (ASGR).
